(19) United States
(12) Patent Application Publication
(10) Pub. No.: US 2006/0084697 A1
(43) Pub. Date: Apr. 20, 2006

(54) Pi3k antagonists as radiosensitizers

(76) Inventors: Dennis E. Hallahan, Nashville, TN (US); Jiahuai Tan, Nashville, TN (US)

Correspondence Address: JENKINS, WILSON & TAYLOR, P. A., 3100 TOWER BLVD, SUITE 1200, DURHAM, NC, 27707, US

(21) Appl. No.: 10/523,948

(22) PCT Filed: Aug. 8, 2003
(86) PCT No.: PCT/US03/25015

§ 371 Date: Oct. 17, 2005

Related U.S. Application Data
(60) Provisional application No. 60/401,864, filed on Aug. 8, 2002.

(51) Int. Cl. A61K 31/7048; A61K 31/366; A61K 31/353

(52) U.S. Cl. 514/453; 514/27; 514/456

(57) ABSTRACT

A method for increasing radiosensitivity of a target tissue in a subject via administration of a PI3K antagonist to a target tissue in a subject. Also provided are methods for suppressing tumor growth and for inhibiting tumor blood vessel growth via administration of a PI3K antagonist.

### CROSS REFERENCE TO RELATED APPLICATIONS

This application is based on and claims priority to U.S. Provisional Application Ser. No. 60/401,864, filed Aug. 8, 2002, herein incorporated by reference in its entirety.

### GRANT STATEMENT

This work was supported by grants R01-CA58508, R01-CA70937, R01-CA89674, R21-CA89888, P30-CA68485, and P50-CA90949 from the U.S. National Institutes of Health (NIH). Thus, the U.S. government has certain rights in the presently claimed subject matter.

### TECHNICAL FIELD

The presently claimed subject matter generally relates to methods for enhancing radiotherapy via inhibition of PI3K signaling. More particularly, the methods of the presently claimed subject matter involve administration of a PI3K antagonist to a target tissue in a subject, whereby the radiosensitivity of a target tissue in a subject is increased.

### TABLE OF ABBREVIATIONS

Akt—protein kinase B
Bad—a member of the Bcl-2 family of apoptosis regulators
Bcl-2—a family of apoptosis regulatory molecules
BFGF—basic fibroblast growth factor
βPDGFRs—platelet-derived growth factor beta receptors
C57BL/6J—a strain or mouse available from the Jackson Laboratory, Bar Harbor, Me., United States of America
CEA—carcinoembryonic antigen
DMEM—Dulbecco's Modified Eagle Medium
DMSO—dimethylsulfoxide
EDTA—ethylenediaminetetra-acetic acid
EGF—epidermal growth factor
EGFR—epidermal growth factor receptor
FGFR—fibroblast growth factor receptor
Flk-1—a receptor for VEGF
g—gram
GBM—glioblastoma multiforme
GFP—green fluorescent protein
GL261—a glioblastoma cell line
GP—glycoprotein
Gy—Grays
H&E—hematoxylin and eosin
HIF-1α—hypoxia-inducible factor-1 alpha
HUVEC—human umbilical vein endothelial cell
¹²⁵I—iodine 125
¹³¹I—iodine 131
ICAM-1—intracellular adhesion molecule-1
IFN-α—interferon-alpha
IFN-γ—interferon-gamma
IGF-1—insulin-like growth factor-I
IgG—immunoglobulin G
IL—interleukin
IMRT—intensity modulated radiation therapy
kVp—kilovolt peak
L—liter
LLC—Lewis lung carcinoma cell line
M—molar
M-CSF—macrophage colony stimulating factor
MHz—megahertz
NIH—National Institutes of Health
³²P—phosphorus 32
PAGE—polyacrylamide gel electrophoresis
PBS—phosphate buffered saline
PBST—phosphate buffered saline with Triton X-100
PDGF—platelet derived growth factor
PDGFR—platelet derived growth factor receptor
pfu—plaque-forming unit(s)
PI3K—phosphatidylinositol 3-kinase
PKB—protein kinase B
PMSF—phenylmethylsulphonyl fluoride
RTK—receptor tyrosine kinase
SDS—sodium dodecyl sulfate
Seg-1—a human adenocarcinoma cell line
Ser—serine
SQ20B—a radioresistant squamous cell carcinoma line
T98—a human melanoma cell line
Tm—melting temperature
TNF—tumor necrosis factor
U1—a human melanoma cell line
U87—a human glioblastoma cell line
VEGF—vascular endothelial growth factor
vWf—von Willebrand factor

### BACKGROUND ART

Cells respond to external stimuli in a number of ways, including by proliferating, differentiating, surviving, or dying. One class of mediators of such responses is the receptor tyrosine kinases (RTKs). RTK activity is initiated by the binding of a ligand (e.g., a growth factor) to the extracellular domain of the RTK, which in turn induces autophosphorylation of the tyrosine kinase domain located within the cell. Autophosphorylation of an RTK leads to the activation of different intracellular pathways through which the signal generated by the binding of the ligand is transmitted. The activities of RTKs are very tightly regulated, since abnormal RTK signal transduction can lead to aberrant cellular proliferation, tumor formation, and cancer.

One current approach to treating tumor formation and cancer in patients is the use of localized ionizing radiation. Radiation causes rapidly proliferating cells, such as tumor and cancer cells, to undergo cell death by apoptosis, both in vivo and in vitro (Antonakopoulos et al., 1994; Li et al., 1994; Mesner et al., 1997). Current radiation therapy is frequently unsuccessful at completely eradicating cancer cells from a patient, however. This is true for at least two reasons. One reason cancer can recur is that it is often not possible to deliver a sufficiently high dose of local radiation to kill tumor cells without concurrently creating an unacceptably high risk of damage to the surrounding normal tissue. Another reason is that tumors show widely varying susceptibilities to radiation-induced cell death. Thus, the inability of local radiation to control tumor growth is a significant clinical outcome leading to unsuccessful cancer therapy (Lindegaard et al., 1996; Suit, 1996; Valter et al., 1999).

One obstacle to designing effective radiotherapy is that there is a poor correlation between cellular responses to ionizing radiation in vitro and in vivo. For example, glioblastoma multiforme (GBM) is insensitive to radiation treatment, and has a universally fatal clinical outcome in both children and adults (Walker et al., 1980; Wallner et al., 1989; Packer, 1999). In vitro studies, however, show that human GBM cell lines exhibit radiosensitivity that is similar to that seen in cell lines derived from more curable human tumors (Allam et al., 1993; Taghian et al., 1993). In accord with the clinical data, the use of in vivo animal models has shown that GBM tumors in vivo are much more radioresistant than the cell lines used to produce them are in vitro (Baumann et al., 1992; Allam et al., 1993; Taghian et al., 1993; Advani et al., 1998; Staba et al., 1998). Thus, the inability to predict the radiosensitivity of a tumor in vivo based upon in vitro experimentation continues to be a significant obstruction to the successful design of radiotherapy treatments of human cancers.

Tumor cells could show enhanced radiosensitivity in vitro compared to in vivo due to the absence of an angiogenic support network in vitro, the presence of which appears to contribute to a tumor's radioresistance in vivo. The response of tumor microvasculature to radiation is dependent upon the dose and time interval after treatment (Kallman et al., 1972; Song et al., 1972; Hilmas & Gillette, 1975; Johnson, 1976; Yamaura et al., 1976; Ting et al., 1991). Tumor blood flow decreases when high doses of radiation in the range of 20 Grays (Gy) to 45 Gy are used (Song et al., 1972). In contrast, blood flow increases when relatively low radiation doses, for example below 500 rads, are administered (Kallman et al., 1972; Hilmas & Gillette, 1975; Johnson, 1976; Yamaura et al., 1976; Gorski et al., 1999). In irradiated mouse sarcomas, for example, blood flow increased during the 3 to 7 days immediately following irradiation (Kallman et al., 1972). Thus, the microvasculature might serve to protect tumor cells from radiation-induced cell death.

RTK activation has been shown to enhance the viability of endothelial cells, such as are found in blood vessels, in response to irradiation (Gorski et al., 1999; Geng et al., 2001; Paris et al., 2001). Vascular endothelial growth factor (VEGF) could be involved in this effect. VEGF is a potent angiogenic growth factor that normally acts directly on vascular endothelium to promote the survival of newly formed vessels (Alon et al., 1995; McMahon, 2000). VEGF has also been implicated in tumor proliferation (Bell et al., 1999), and several transformed cell lines express unusually high levels of VEGF (Kieser et al., 1994; Grugel et al., 1995; Graeven et al., 1999). In addition, elevated VEGF expression is clinically relevant as it is associated with worsened prognosis (Valter et al., 1999).

Elevated VEGF levels also correlate with radiation stress and radiotherapy resistance (Shintani et al., 2000). For example, VEGF expression is elevated in such radioresistant tumors as malignant glioma and melanoma (Liu et al., 1995). Interfering with VEGF signal transduction increases the in vitro radiosensitivity of glioblastoma and melanoma tumor models (Geng et al., 2001). These data suggest a role for VEGF in promoting cellular survival following radiotherapy. The mechanisms by which VEGF exerts this protective effect have not been elucidated, however.

Both in vitro and in vivo experiments have suggested that VEGF expression is induced when cells or tumors are exposed to ionizing radiation (Katoh et al., 1995; Gorski et al., 1999). For example, when growing Lewis lung carcinoma (LLC) cells are treated in vitro with different doses of irradiation, VEGF levels showed a dose-dependent increase within 24 hours of treatment (Gorski et al., 1999). Several other human tumor cell lines also showed an increase in VEGF expression after in vitro exposure to radiation, including Seg-1 (esophageal adenocarcinoma), SQ20B (a radioresistant squamous cell carcinoma line), U1 (melanoma), and T98 and U87 (glioblastoma; Gorski et al., 1999). Tumors produced in vivo by implanting LLC, Seg-1, or SQ20B cells into mice also showed enhanced VEGF expression after exposure to radiation (Gorski et al., 1999).

The induction of VEGF expression is associated with increased radioresistance of these cells and tumors. Neutralizing antibodies to VEGF, a soluble extracellular component of the Flk-1 receptor (one of three VEGF receptors so far identified), and a Flk-1-specific inhibitor are all able to eliminate this resistance phenotype both in vitro and in vivo, presumably by interfering with the interaction of VEGF with its receptor(s) (Gorski et al., 1999; Geng et al., 2001). Currently, however, effective strategies for enhancing the radiosensitivity of tumors in vivo by interfering with VEGF signal transduction are not available.

Protein kinase B, also called Akt, is another example of an RTK that is involved in promoting cellular survival. Akt is activated by several different growth factors, including insulin-like growth factor-I (IGF-I), epidermal growth factor (EGF), basic fibroblast growth factor (bFGF), insulin, interleukin-6 (IL6), and macrophage colony stimulating factor (M-CSF; Datta et al., 1999). In vitro studies have shown that constitutively active Akt can block stimulus-induced cell death by phosphorylating mediators of apoptosis (Datta et al., 1999). These mediators include Bad, a member of the Bcl-2 family, and caspase-9. Phosphorylation of Bad or caspase-9 results in the inhibition of its pro-apoptotic functions (Datta et al., 1997; Cardone et al., 1998). In addition, other members of the apoptotic machinery, as well as several transcription factors, contain the Akt consensus phosphorylation site (Datta et al., 1999). Taken together, these observations strongly suggest that Akt might play a role in enhancing cellular survival in response to apoptotic stimuli.

Akt can be activated through the phosphatidylinositol 3-kinase (PI3K) pathway (Fantl et al., 1993), although PI3K-independent activation of Akt also occurs (Cheng et al., 1996; Shaw et al., 1998; Yano et al., 1998; Filippa et al., 1999; Ushio-Fukai et al., 1999). PI3K catalyzes the addition of a phosphate group to the inositol ring of phosphoinositides normally present in the plasma membrane of cells (Wymann & Pirola, 1998). The products of these reactions, including phosphatidyl-4,5-bisphosphate and phosphatidyl-3,4,5-trisphosphate, are potent second messengers of several RTK signals (Cantley, 2002). In vitro studies have indicated that Akt and PI3K are involved in growth factor-mediated survival of various cell types (Datta et al., 1999), including neural cells (Yao & Cooper, 1995; Dudek et al., 1997; Weiner & Chun, 1999), fibroblasts (Kauffmann-Zeh et al., 1997; Fang et al., 2000), and certain cells of hematopoietic origin (Katoh et al., 1995; Kelley et al., 1999; Somervaille et al., 2001).

Recent evidence suggests that the cellular survival pathways involving VEGF and Akt/PI3K might overlap. For example, neovascular endothelial cells upregulate the expression of platelet-derived growth factor β receptors (βPDGFRs) during such processes as wound healing, inflammation, and glioma tumorigenesis (Wang et al., 1999). Treatment of these cells with PDGF increases the expression of VEGF, and this increase is dependent on PI3K (Wang et al., 1999). Akt and PI3K are also involved in the VEGF-induced upregulation of intracellular adhesion molecule-1 (ICAM-1; Radisavljevic et al., 2000). Finally, Akt has been shown to be involved in tumor-induced angiogenesis, an effect mediated through VEGF in conjunction with hypoxia-inducible factor-1α (HIF-1α; Gao et al., 2002). However, the involvement of the Akt/PI3K pathway in the generation of downstream signals for cellular survival induced by VEGF has not been established in vivo.

Thus, there exists a long-felt need in the art for effective therapies for treating tumors that are resistant to conventional therapies, including radiotherapy. To address this need, the presently claimed subject matter provides a method for enhancing the radiosensitivity of cells in a target tissue via administration of a PI3K antagonist.

### SUMMARY

The presently claimed subject matter provides a method for increasing the radiosensitivity of a target tissue in a subject. In one embodiment, the method comprises administering a PI3K antagonist to a subject, whereby the radiosensitivity of the target tissue is increased.

The presently claimed subject matter also provides a method for suppressing tumor growth in a subject, the method comprising: (a) administering a PI3K antagonist to a subject bearing a tumor to increase the radiosensitivity of the tumor; and (b) treating the tumor with ionizing radiation, whereby tumor growth is suppressed.

The presently claimed subject matter also provides a method for inhibiting tumor blood vessel growth, the method comprising: (a) administering a PI3K antagonist to a subject bearing a tumor to increase the radiosensitivity of tumor blood vessels; and (b) treating the tumor with ionizing radiation, whereby tumor blood vessel growth is inhibited. A PI3K antagonist can also be administered after irradiation as maintenance therapy for the prevention of vascular regrowth.

The methods of the presently claimed subject matter are useful for radiosensitizing target tissues, suppressing tumor growth, and inhibiting tumor vascularization in mammalian subjects including but not limited to human subjects. The methods can be used for any suitable target tissue, including but not limited to vascular tissue, vascular endothelium, and tumors such as radiation resistant tumors.

A PI3K antagonist can be administered as a minimally therapeutic dose, although higher doses can be used as well. Representative doses of ionizing radiation include but are not limited to a subtherapeutic dose and a therapeutic dose.

Representative PI3K antagonists include LY294002 and wortmannin; small molecules that specifically bind PI3K, including, but not limited to SU6668, SU11248, and Genistein; small molecules that inhibit the activation of downstream mediators of PI3K signaling; a dominant negative PI3K polypeptide; and neutralizing antibodies to PI3K.

Accordingly, it is an object of the presently claimed subject matter to provide novel methods for radiosensitizing target tissues, including tumors. This and other objects are achieved in whole or in part by the presently claimed subject matter.

An object of the presently claimed subject matter having been stated above, other objects and advantages of the presently claimed subject matter will become apparent to those of ordinary skill in the art after a study of the following description of the presently claimed subject matter and non-limiting Examples.

### DETAILED DESCRIPTION

I. Radiosensitivity

In one embodiment, a novel method for increasing the radiosensitivity of a target tissue in a subject via administration of a PI3K antagonist is provided. The method comprises administering a PI3K antagonist to the subject, whereby the radiosensitivity of a target tissue is increased. The presently claimed subject matter also provides a method for suppressing tumor growth in a subject. The method comprises: (a) administering a PI3K antagonist to a subject bearing a tumor to increase the radiosensitivity of the tumor; and (b) treating the tumor with ionizing radiation, whereby tumor growth is suppressed. The presently claimed subject matter also provides a method for inhibiting tumor blood vessel growth. The method comprises: (a) administering a PI3K antagonist to a subject bearing a tumor to increase the radiosensitivity of tumor blood vessels; and (b) treating the tumor with ionizing radiation, whereby tumor blood vessel growth is inhibited.

The term “radiosensitivity” as used herein to describe a target tissue refers to a quality of susceptibility to treatment using ionizing radiation. This susceptibility can result from direct effects of the radiation on the cells of the target tissue themselves. For example, radiation can cause the cells of the target tissue to undergo apoptosis as a result of either DNA damage or another cell autonomous mechanism. Alternatively, radiosensitivity can result from indirect effects, such as effects on the microenvironment of the cells of the target tissue, for example, on the blood vessels supplying nutrients and oxygen to the target tissue. Thus, radiotherapy can be used to suppress the growth of a radiosensitive target tissue.

Radiosensitivity can be quantified by determining a minimal amount of ionizing radiation that can be used to delay target tissue growth. Thus, the term “radiosensitivity” refers to a quantitative range of radiation susceptibility.

The term “target tissue” refers to any cell or group of cells present in a subject. This term includes single cells and populations of cells. The term includes but is not limited to cell populations comprising glands and organs such as skin, liver, heart, kidney, brain, pancreas, lung, stomach, and reproductive organs. It also includes but is not limited to mixed cell populations such as bone marrow. Further, it includes but is not limited to such abnormal cells as neoplastic or tumor cells, whether individually or as a part of solid or metastatic tumors. The term “target tissue” as used herein additionally refers to an intended site for accumulation of a ligand following administration to a subject. For example, the methods of the presently claimed subject matter employ a target tissue comprising a tumor.

The term “suppressing tumor growth” refers to an increase in a duration of time required for a tumor to grow a specified amount. For example, treatment can extend the time required for a tumor to increase in volume 3-fold relative to an initial day of measurement (day 0) or the time required to grow to a volume of 1 cm³.

The terms “radiation resistant tumor” and “radioresistant tumor” each generally refer to a tumor that is substantially unresponsive to radiotherapy when compared to other tumors. Representative radiation resistant tumor models include glioblastoma multiforme and melanoma.

The term “increase” as used herein to refer to a change in radiosensitivity of a tumor refers to change that renders a tumor more susceptible to destruction by ionizing radiation. Alternatively stated, an increase in radiosensitivity refers to a decrease in the minimal amount of ionizing radiation that effectively suppresses tumor growth. An increase in radiosensitivity can also comprise suppressed tumor growth or inhibited tumor blood vessel growth when a PI3K antagonist is administered with radiation as compared to a same dose of radiation alone. In one example, an increase in radiosensitivity refers to an increase of at least about 2-fold, in another example to an increase of at least about 5-fold, and in still another example an increase of at least 10-fold. In one embodiment of the presently claimed subject matter, an increase in radiosensitivity comprises a transformation of a radioresistant tumor to a radiosensitive tumor.

The methods of the presently claimed subject matter are useful for increasing the radiosensitivity of a target tissue, for suppressing tumor growth, and/or for inhibiting tumor blood vessel growth in any subject. Thus, the term “subject” as used herein includes any vertebrate species, for example, warm-blooded vertebrates such as mammals and birds. More particularly, the methods of the presently claimed subject matter are contemplated for the treatment of tumors in mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants and livestock (such as cattle, oxen, sheep, giraffes, deer, goats, bison, and camels), and horses. Also contemplated is the treatment of birds, including those kinds of birds that are endangered or kept in zoos, as well as fowl, and more particularly domesticated fowl or poultry, such as turkeys, chickens, ducks, geese, guinea fowl, and the like, as they are also of economic importance to humans.

The term “tumor” as used herein encompasses both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas. The term “tumor” also encompasses radioresistant tumors, including radioresistant variants of the any of the tumor listed above.

The term “about”, as used herein when referring to a measurable value such as an amount of weight, time, dose (e.g. radiation dose), etc. is meant to encompass in one example variations of ±20% or ±10%, in another example ±5%, in another example ±1%, and in yet another example ±0.1% from the specified amount, as such variations are appropriate to perform the disclosed method.

II. PI3K Antagonists

The presently claimed subject matter provides methods for increasing the radiosensitivity of a target tissue via administration of a PI3K antagonist. Any suitable PI3K antagonist can be used in accordance with the methods of the presently claimed subject matter, wherein the antagonist has a capacity to increase the radiosensitivity of a target tissue. In one embodiment, a PI3K antagonist also shows anti-angiogenic activity or angiostatic activity.

The term “PI3K antagonist” as used herein refers to a molecule or other chemical entity having a capacity for specifically binding to PI3K to thereby inhibit a PI3K biological activity. The term “PI3K antagonist” as used herein also refers to a molecule or other chemical entity having the capacity for preventing Akt activation, including but not limited to broad-spectrum receptor tyrosine kinase inhibitors that prevent radiation-induced activation of the PI3K/Akt signaling pathway. Stated another way, an aspect of the presently claimed subject matter pertains to the observation that broad-spectrum receptor tyrosine kinase inhibitors prevent radiation-induced activation of the PI3K/Akt signaling pathway. PI3K antagonists include but are not limited to small molecule inhibitors, neutralizing antibodies, and soluble PI3K polypeptides.

The term “binding” refers to an affinity between two molecules, for example, an inhibitor and a target molecule. As used herein, “specific binding” means a preferential binding of one molecule for another in a mixture of molecules. The binding of an inhibitor to a target molecule can be considered specific if the binding affinity is about 1×10⁴ M⁻¹ to about 1×10⁶ M⁻¹ or greater.

II.A. Small Molecules

The term “small molecule” as used herein refers to a compound, for example an organic compound, with a molecular weight of in one example less than about 1,000 daltons, in another example less than about 750 daltons, in another example less than about 600 daltons, and in still another example less than about 500 daltons. A small molecule also in one example has a computed log octanol—water partition coefficient in the range of about −4 to about +14, and in another example in the range of about −2 to about +7.5.

Wortmannin and LY294002 are small molecule antagonists of PI3K (Wang et al., 1999; Radisavljevic et al., 2000; Brognard et al., 2001). Preparation/synthesis schemes and structural information for wortmannin and LY294002 are disclosed in Powis et al., 1994 and U.S. Pat. No. 5,480,906 and in Vlahos et al., 1994, respectively, and references therein. SU6668 (3-[2,4-dimethyl-5-(2-oxo-1,2-dihydro-indol-3-ylidenemethyl)-1H-pyrrol-3-yl]-propionic acid), SU11248, and Genistein (4′,5,7-trihydroxyisoflavone) are small molecule antagonists of PI3K. That is, these broad-spectrum receptor tyrosine kinase inhibitors have been observed to prevent radiation-induced activation of the PI3K/Akt signaling pathway, in accordance with the presently claimed subject matter.

Further, broad-spectrum RTK inhibitors that attenuate radiation-induced activation of Akt include SU6668 and SU11248. The broad-spectrum tyrosine kinase inhibitor Genistein also attenuates Akt activation in irradiated vascular endothelium.

II.B. Soluble PI3K Polypeptides

The term “soluble PI3K polypeptide” refers to a PI3K polypeptide that inhibits PI3K signaling. In one embodiment, a soluble PI3K polypeptide comprises a truncated PI3K polypeptide having an ability to bind to mediators of signal transduction upstream of PI3K, to heterodimerize with native PI3K, to bind to PI3K thereby inactivating PI3K, or a combination thereof. The truncated soluble form of PI3K can also display inhibition of PI3K signaling and/or suppression of tumor growth and tumor blood vessel growth. Although it is not applicants' desire to be bound by any particular theory of operation, it is believed that soluble PI3K binds to and sequesters mediators of signal transduction upstream of PI3K, thereby limiting the availability of these mediators to pass on their proliferative and/or survival signals. Although it is not applicants' desire to be bound by any particular theory of operation, it is believed that soluble PI3K also functions as a dominant negative receptor by forming inactive heterodimers with native PI3K or by interacting with substrate-binding region of PI3K.

The terms “nucleic acid molecule” and “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, or “mRNA”. Nucleic acids can be synthesized, or can be derived from any biological source, including any organism.

The term “substantially identical”, as used herein to describe a degree of similarity between nucleotide sequences, refers to two or more sequences that have in one embodiment at least about least 60%, in another embodiment at least about 70%, in another embodiment at least about 80%, in another embodiment about 90% to about 99%, in another embodiment about 95% to about 99%, and in still another embodiment about 99% nucleotide identity, when compared and aligned for maximum correspondence, as measured using a sequence comparison algorithm (described herein below under the heading “Nucleotide and Amino Acid Sequence Comparisons”) or by visual inspection. In one embodiment, the substantial identity exists in nucleotide sequences of at least about 100 residues, in another embodiment in nucleotide sequences of at least about 150 residues, and in yet another embodiment in nucleotide sequences comprising a full length coding sequence. The term “full length”, as used herein refers to a complete open reading frame encoding a functional soluble PI3K polypeptide.

In one aspect, substantially identical sequences can comprise polymorphic sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise a single base change.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

In one embodiment, a nucleotide sequence employed for hybridization studies or assays includes probe sequences that are complementary to or mimic at least an about 14 to 40 nucleotide sequence of a nucleic acid molecule of the presently claimed subject matter. in another embodiment, probes comprise 14 to 20 nucleotides, or even longer where desired, such as 30, 40, 50, 60, 100, 200, 300, or 500 nucleotides or up to the full length of any one of the sequences of the presently claimed subject matter. Such probes can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent hybridization and wash conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tm) for the specific sequence at a defined ionic strength and pH. Typically, under “stringent conditions” a probe will hybridize specifically to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., 1989, for a description of SSC buffer. Often, a high stringency wash is preceded by a low stringency wash to remove background probe signal. An example of medium stringency wash conditions for a duplex of more than about 100 nucleotides is 15 minutes in 1×SSC at 45° C. An example of low stringency wash for a duplex of more than about 100 nucleotides is 15 minutes in 4× to 6×SSC at 40° C. For short probes (e.g., about 10 to 50 nucleotides), stringent conditions typically involve salt concentrations of less than about 1M Na+ ion, typically about 0.01 to 1M Na+ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the presently claimed subject matter: a probe nucleotide sequence in one example hybridizes to a target nucleotide sequence in 7% sodium dodecyl sulfate (SDS), 0.5M NaPO₄, 1 mM EDTA at 50° C. followed by washing in 2×SSC, 0.1% SDS at 50° C.; in another example, a probe and target sequence hybridize in 7% sodium dodecyl sulfate (SDS), 0.5M NaPO₄, 1 mM EDTA at 50° C. followed by washing in 1×SSC, 0.1% SDS at 50° C.; in another example, a probe and target sequence hybridize in 7% sodium dodecyl sulfate (SDS), 0.5M NaPO₄, 1 mM EDTA at 50° C. followed by washing in 0.5×SSC, 0.1% SDS at 50° C.; in another example, a probe and target sequence hybridize in 7% sodium dodecyl sulfate (SDS), 0.5M NaPO₄, 1 mM EDTA at 50° C. followed by washing in 0.1×SSC, 0.1% SDS at 50° C.; in another example, a probe and target sequence hybridize in 7% sodium dodecyl sulfate (SDS), 0.5M NaPO₄, 1 mM EDTA at 50° C. followed by washing in 0.1×SSC, 0.1% SDS at 65° C.

A further indication that two nucleic acid sequences are substantially identical is that the proteins encoded by the nucleic acids are substantially identical, share an overall three-dimensional structure, or are biologically functional equivalents. These terms are defined further under the heading “Polypeptides” herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues (Ohtsuka et al., 1985; Batzer et al., 1991; Rossolini et al., 1994).

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising in one example about 8 or more deoxyribonucleotides or ribonucleotides, in another example 10-20 nucleotides, and in still another example 20-30 nucleotides of a selected nucleic acid molecule. The primers of the presently claimed subject matter encompass oligonucleotides of sufficient length and appropriate sequence so as to provide initiation of polymerization on a nucleic acid molecule of the presently claimed subject matter.

The term “elongated sequence” refers to a sequence comprising additional nucleotides (or other analogous molecules) incorporated into and/or at either end of a nucleic acid. For example, a polymerase (e.g., a DNA polymerase) can add sequences at the 3′ terminus of a nucleic acid molecule. In addition, a nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. An example of a complementary nucleic acid segment is an antisense oligonucleotide.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The presently claimed subject matter also encompasses chimeric genes comprising a nucleotide sequence encoding a soluble PI3K polypeptide. The term “chimeric gene”, as used herein, refers to a gene comprising a heterologous promoter region operatively linked to a nucleotide sequence encoding a soluble PI3K polypeptide.

The term “operatively linked”, as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

The term “heterologous”, as used herein to refer to a promoter or any other nucleic acid, refers to a sequence that originates from a source foreign to an intended host cell or, if from the same source, is modified from its original form. Thus, a heterologous nucleic acid in a host cell includes a gene that is endogenous to the particular host cell but has been modified, for example by mutagenesis or by isolation from native cis-regulatory sequences. The term “heterologous nucleic acid” also includes non-naturally occurring multiple copies of a native nucleotide sequence. The term “heterologous nucleic acid” also encompasses a nucleic acid that is incorporated into a host cell's nucleic acids, however at a position wherein such nucleic acids are not ordinarily found. A representative heterologous nucleic acid comprises a recombinant nucleic acid, as described further herein below.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, which enable its replication in a host cell.

The term “vector” is used herein to refer to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors include plasmids, cosmids, and viral vectors. A vector can also mediate recombinant production of a soluble PI3K polypeptide.

The term “construct”, as used herein to describe an expression construct, refers to a vector further comprising a nucleotide sequence operatively inserted with the vector, such that the nucleotide sequence is expressed.

The terms “recombinantly expressed” or “recombinantly produced” are used interchangeably to generally refer to the process by which a polypeptide encoded by a recombinant nucleic acid is produced.

The term “heterologous expression system” refers to a host cell comprising a heterologous nucleic acid and the polypeptide encoded by the heterologous nucleic acid. For example, a heterologous expression system can comprise a host cell transfected with a construct comprising a recombinant nucleic acid, or a cell line produced by introduction of heterologous nucleic acids into a host cell genome.

Nucleic acids of the presently claimed subject matter can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions is also known in the art as exemplified by publications. See e.g., Sambrook et al., 1989; Silhavy et al., 1984; Glover & Hames, 1995; and Ausubel, 1995.

In one embodiment, the presently claimed subject matter provides a viral construct encoding soluble PI3K polypeptides. The presently claimed subject matter also provides a recombinantly expressed and isolated soluble PI3K polypeptide.

The term “substantially identical”, as used herein to describe a level of similarity between a soluble PI3K polypeptide and a protein substantially identical to a soluble PI3K polypeptide, refers to a sequence that is at least 35% identical to a given sequence when compared over the full length of a soluble PI3K protein. In one embodiment, a protein substantially identical to soluble PI3K comprises an amino acid sequence that is at least about 35% to about 45% identical to a given sequence, in another embodiment at least about 45% to about 55% identical to a given sequence, and in still another embodiment at least about 55% to about 65% identical to a given sequence when compared over the full length of a soluble PI3K polypeptide. Methods for determining percent identity are defined herein below under the heading “Nucleotide and Amino Acid Sequence Comparisons”.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites (Barton, 1998; Saqi et al., 1999; Henikoff et al., 2000; Huang et al., 2000).

Substantially identical proteins also include proteins comprising amino acids that are functionally equivalent to amino acids of any one of the provided sequences. The term “functionally equivalent” in the context of amino acid sequences is known in the art and is based on the relative similarity of the amino acid side-chain substituents (Henikoff & Henikoff, 2000). Relevant factors for consideration include side-chain hydrophobicity, hydrophilicity, charge, and size. For example, arginine, lysine, and histidine are all positively charged residues; alanine, glycine, and serine are all of similar size; and phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art. See e.g., Kyte & Doolittle, 1982. It is known that certain amino acids can be substituted for other amino acids having a similar hydropathic index or score and still retain a similar biological activity. In making changes based upon the hydropathic index, one example involves the substitution of amino acids whose hydropathic indices are within ±2 of the original value, another example involves those that are within ±1 of the original value, and yet another example involves those within ±0.5 of the original value.

It is also understood in the art that the substitution of like amino acids can be made effectively based on hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, one example involves the substitution of amino acids whose hydrophilicity values are within ±2 of the original value, another example involves those that are within ±1 of the original value, and still another example involves those within ±0.5 of the original value.

The term “substantially identical” also encompasses polypeptides that are biologically functional equivalents. The term “functional” includes activity of a soluble PI3K polypeptide in inhibiting PI3K signaling and increasing radiosensitivity of a tumor, as described herein. Methods for assessing a radiosensitizing function are described herein.

The presently claimed subject matter also provides functional fragments of a soluble PI3K polypeptide. Such functional portion need not comprise all or substantially all of the amino acid sequence of an extracellular portion of a native PI3K gene product.

The presently claimed subject matter also includes functional polypeptide sequences that are longer sequences than that of an extracellular portion of a native soluble PI3K polypeptide. For example, one or more amino acids can be added to the N-terminus or C-terminus of a soluble PI3K polypeptide. Methods of preparing such proteins are known in the art.

Nucleotide and Amino Acid Sequence Comparisons. The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman, 1981, by the homology alignment algorithm of Needleman & Wunsch, 1970, by the search for similarity method of Pearson & Lipman, 1988, by computerized implementations of these algorithms (GAP, BESTFIT, FASTA, and TFASTA in the GCG® WISCONSIN PACKAGE® available from Accelrys Inc., San Diego, Calif., United States of America), or by visual inspection. See generally, Ausubel, 1995.

One example of an algorithm for determining percent sequence identity and sequence similarity is the BLAST algorithm, which is described by Altschul et al., 1990. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (Henikoff & Henikoff, 1992).

In addition to calculating percent sequence identity, the BLAST algorithm also performs a statistical analysis of the similarity between two sequences (Karin & Altschul, 1993). One measure of similarity provided by the BLAST algorithm is the smallest sum probability (P(N)), which provides an indication of the probability by which a match between two nucleotide or amino acid sequences would occur by chance. For example, in one example a test nucleic acid sequence is considered similar to a reference sequence if the smallest sum probability in a comparison of the test nucleic acid sequence to the reference nucleic acid sequence is less than about 0.1, in another example less than about 0.01, and in yet another example less than about 0.001.

II.C. PI3K Antibodies

The presently claimed subject matter further provides a PI3K antagonist comprising an antibody that specifically binds PI3K. Optionally, a PI3K antagonist can further comprise a carrier for sustained bioavailability of the antibody at a tumor. The disclosure herein reveals that a prolonged or sustained release of PI3K antagonist is optionally employed to enhance the therapeutic effect of combined PI3K antagonism and radiation.

The term “antibody” indicates an immunoglobulin protein, or functional portion thereof, including a polyclonal antibody, a monoclonal antibody, a chimeric antibody, a hybrid antibody, a single chain antibody (e.g., a single chain antibody represented in a phage library), a mutagenized antibody, a humanized antibody, and antibody fragments that comprise an antigen binding site (e.g., Fab and Fv antibody fragments). In one embodiment, an antibody of the presently claimed subject matter is a monoclonal antibody.

Techniques for preparing and characterizing antibodies are known in the art. See e.g., Harlow & Lane, 1988 and U.S. Pat. Nos. 4,196,265; 4,946,778; 5,091,513; 5,132,405; 5,260,203; 5,677,427; 5,892,019; 5,985,279; 6,054,561. Single chain antibodies can be identified by screening a phage antibody library, for example as described by U.S. Pat. Nos. 6,174,708; 6,057,098; 5,922,254; 5,840,479; 5,780,225; 5,702,892; and 5,667,988.

An antibody of the presently claimed subject matter can further be mutagenized or otherwise modified to improve antigen binding and/or antibody stability. For example, to prevent undesirable disulfide bond formation, a nucleotide sequence encoding the variable domain of an antibody or antibody fragment can be modified to eliminate at least one of each pair of codons that encode cysteines for disulfide bond formation. Recombinant expression of the modified nucleotide sequence, for example in a prokaryotic expression system, results in an antibody having improved stability. See U.S. Pat. No. 5,854,027.

The term “sustained bioavailability” is used herein to describe a composition comprising a PI3K antagonist and a carrier, whereby the bioavailability of a PI3K antagonist at a tumor site is sufficient to achieve radiosensitization of a tumor. The term “sustained bioavailability” also refers to a bioavailability sufficient to inhibit blood vessel growth within the tumor. The term “sustained bioavailability” encompasses factors including but not limited to sustained release of a PI3K antagonist from a carrier, metabolic stability of a PI3K antagonist, systemic transport of a composition comprising a PI3K antagonist, and effective dose of a PI3K antagonist. Representative approaches for preparing a sustained bioavailability composition are described herein below under the heading “Carriers”.

As disclosed herein, an immediate response of tumor blood vessels to radiation is a decrease in tumor blood flow. This response can diminish administration of an anti-tumor composition (e.g., a PI3K antagonist). Recognizing this response, the disclosure of the presently claimed subject matter provides that sustained bioavailability of a PI3K antagonist, for example by selection of a carrier and administration regimen that achieve sustained bioavailability, can improve anti-tumor activity. One example of carrier comprises a gene therapy vector encoding a PI3K antagonist.

A method comprising a carrier or administration approach for sustained bioavailability can also improve therapies directed toward modulation of other components of the PI3K signaling pathway. For example, treatments that employed direct administration of an anti-VEGF antibody (in the absence of a carrier or administration regimen for sustained bioavailability of the antibody at the tumor) showed variable anti-tumor efficacy in different tumor types (Gorski et al., 1999; Lee et al., 2000). In addition, therapeutic effects were not observed when sub-therapeutic or minimally therapeutic doses of the anti-VEGF antibody were used. Thus, the presently claimed subject matter further provides an improved method for inhibiting tumor growth, the method comprising administration of a gene therapy vector encoding an inhibitor of VEGF signaling, whereby bioavailability of the inhibitor at a tumor is sustained, and whereby tumor growth delay is improved.

II.D. Sustained Bioavailability

The term “sustained bioavailability” is used herein to describe a composition comprising a PI3K antagonist and a carrier, whereby the bioavailability of a PI3K antagonist at a tumor site is sufficient to achieve radiosensitization of a tumor. The term “sustained bioavailability” also refers to a bioavailability sufficient to inhibit blood vessel growth within the tumor. The term “sustained bioavailability” encompasses factors including but not limited to sustained release of a PI3K antagonist from a carrier, metabolic stability of a PI3K antagonist, systemic transport of a composition comprising a PI3K antagonist, and effective dose of a PI3K antagonist. Representative approaches for preparing a sustained bioavailability composition are described herein below under the heading “Carriers”.

As disclosed herein, an immediate response of tumor blood vessels to radiation is a decrease in tumor blood flow. This response can diminish administration of an anti-tumor composition (e.g., a PI3K antagonist). Recognizing this response, the disclosure of the presently claimed subject matter provides that sustained bioavailability of a PI3K antagonist, for example by selection of a carrier and administration regimen that achieve sustained bioavailability, can improve anti-tumor activity. One example of carrier comprises a gene therapy vector encoding a PI3K antagonist.

A method comprising a carrier or administration approach for sustained bioavailability can also improve therapies directed toward modulation of other components of the PI3K signaling pathway. For example, treatments that employed direct administration of an anti-VEGF antibody (in the absence of a carrier or administration regimen for sustained bioavailability of the antibody at the tumor) showed variable anti-tumor efficacy in different tumor types (Gorski et al., 1999; Lee et al., 2000). In addition, therapeutic effects were not observed when sub-therapeutic or minimally therapeutic doses of the anti-VEGF antibody were used. Thus, the presently claimed subject matter further provides an improved method for inhibiting tumor growth, the method comprising administration of a gene therapy vector encoding an inhibitor of VEGF signaling, whereby bioavailability of the inhibitor at a tumor is sustained, and whereby tumor growth delay is improved.

III. Therapeutic Compositions

In accordance with the methods of the presently claimed subject matter, a composition that is administered to increase the radiosensitivity of a target tissue in a subject comprises: (a) a PI3K antagonist; and (b) a pharmaceutically acceptable carrier. Any suitable carrier that facilitates drug preparation and/or administration can be used.

III.A. Carriers

The carrier can be a viral vector or a non-viral vector. Suitable viral vectors include adenoviruses, adeno-associated viruses (AAVs), retroviruses, pseudotyped retroviruses, herpes viruses, vaccinia viruses, Semiliki forest virus, and baculoviruses. In one embodiment of the presently claimed subject matter, the carrier comprises an adenoviral gene therapy construct that encodes a PI3K antagonist.

Suitable non-viral vectors that can be used to deliver a PI3K antagonist include but are not limited to a plasmid, a nanosphere (Manome et al., 1994; Saltzman & Fung, 1997), a peptide (U.S. Pat. Nos. 6,127,339 and 5,574,172), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al., 1997) and U.S. Pat. Nos. 4,551,482, 5,714,166, 5,510,103, 5,490,840, and 5,855,900), and a polysome (U.S. Pat. No. 5,922,545).

Where appropriate, two or more types of carriers can be used together. For example, a plasmid vector can be used in conjunction with liposomes. Currently, one embodiment of the presently claimed subject matter envisions the use of an adenovirus.

In one embodiment, a composition of the presently claimed subject matter comprises a PI3K antagonist and a carrier to effect sustained bioavailability of the PI3K antagonist following administration to a tumor-bearing subject. The term “sustained bioavailability” is used herein to refer to a bioavailability of a PI3K antagonist sufficient to achieve radiosensitization of a tumor. The term “sustained bioavailability” also refers to a bioavailability of a PI3K antagonist sufficient to inhibit blood vessel growth within a tumor. The term “sustained bioavailability” encompasses factors including but not limited to prolonged release of a PI3K antagonist from a carrier, metabolic stability of a VEGF-R2 inhibitor, systemic transport of a composition comprising a PI3K antagonist, and effective dose of a PI3K antagonist.

Representative compositions for sustained bioavailability of a PI3K antagonist can include but are not limited to polymer matrices, including swelling and biodegradable polymer matrices, (U.S. Pat. Nos. 6,335,035; 6,312,713; 6,296,842; 6,287,587; 6,267,981; 6,262,127; and 6,221,958), polymer-coated microparticles (U.S. Pat. Nos. 6,120,787 and 6,090,925) a polyol:oil suspension (U.S. Pat. No. 6,245,740), porous particles (U.S. Pat. No. 6,238,705), latex/wax coated granules (U.S. Pat. No. 6,238,704), chitosan microcapsules, and microsphere emulsions (U.S. Pat. No. 6,190,700).

One embodiment for sustained bioavailability of a PI3K antagonist comprises a gene therapy construct comprising a gene therapy vector, for example a gene therapy vector described herein below.

Viral Gene Therapy Vectors. In one embodiment, viral vectors of the presently claimed subject matter are disabled; e.g. replication-deficient. That is, they lack one or more functional genes required for their replication, which prevents their uncontrolled replication in vivo and avoids undesirable side effects of viral infection. In one embodiment, all of the viral genome is removed except for the minimum genomic elements required to package the viral genome incorporating the therapeutic gene into the viral coat or capsid. For example, it is desirable to delete all the viral genome except: (a) the Long Terminal Repeats (LTRs) or Inverted Terminal Repeats (ITRs); and (b) a packaging signal. In the case of adenoviruses, deletions are typically made in the E1 region and optionally in one or more of the E2, E3, and/or E4 regions. Other viral vectors can be similarly deleted of genes required for replication. Deletion of sequences can be achieved by recombinant means, for example, involving digestion with appropriate restriction enzymes, followed by religation. Replication-competent self-limiting or self-destructing viral vectors can also be used.

Nucleic acid constructs of the presently claimed subject matter can be incorporated into viral genomes by any suitable means known in the art. Typically, such incorporation is performed by ligating the construct into an appropriate restriction site in the genome of the virus. Viral genomes can then be packaged into viral coats or capsids using any suitable procedure. In particular, any suitable packaging cell line can be used to generate viral vectors of the presently claimed subject matter. These packaging lines complement the replication-deficient viral genomes of the presently claimed subject matter, as they include, for example by incorporation into their genomes, the genes that have been deleted from the replication-deficient genome. Thus, the use of packaging lines allows viral vectors of the presently claimed subject matter to be generated in culture.

Suitable packaging lines for retroviruses include derivatives of PA317 cells, ψ-2 cells, CRE cells, CRIP cells, E-86-GP cells, and 293GP cells. Line 293 cells can be used with adenoviruses and adeno-associated viruses.

Plasmid Gene Therapy Vectors. A soluble PI3K polypeptide can also be encoded by a plasmid. Advantages of a plasmid carrier include low toxicity and easy large-scale production. A polymer-coated plasmid can be delivered using electroporation as described by Fewell et al., 2001. Alternatively, a plasmid can be combined with an additional carrier, for example a cationic polyamine, a dendrimer, or a lipid, that facilitates delivery (Baher et al., 1999; Maruyama-Tabata et al., 2000; Tam et al., 2000).

Liposomes. A PI3K antagonist of the presently claimed subject matter can also be delivered using a liposome. For example, a recombinantly produced soluble PI3K polypeptide can be encapsulated in liposomes. Liposomes can be prepared by any of a variety of techniques that are known in the art. See e.g., Dracopoli et al., 1997; Lasic & Martin, 1995; Janoff, 1999; Gregoriadis, 1993; Betageri et al., 1993.; and U.S. Pat. Nos. 4,235,871; 4,551,482; 6,197,333; and 6,132,766. Temperature-sensitive liposomes can also be used, for example THERMOSOMES™, as disclosed in U.S. Pat. No. 6,200,598. Entrapment of a PI3K antagonist within liposomes of the presently claimed subject matter can be carried out using any conventional method in the art. In preparing liposome compositions, stabilizers such as antioxidants and other additives can be used.

Other lipid carriers can also be used in accordance with the claimed presently claimed subject matter, such as lipid microparticles, micelles, lipid suspensions, and lipid emulsions. See e.g., Labat-Moleur et al., 1996; and U.S. Pat. Nos. 5,011,634; 6,056,938; 6,217,886; 5,948,767; and 6,210,707.

III.B. Targeting Ligands

As desired, a composition of the presently claimed subject matter can include one or more ligands having affinity for a specific cellular marker to thereby enhance delivery of a PI3K antagonist to a tumor in vivo. Ligands include antibodies, cell surface markers, peptides, and the like, which act to home the PI3K antagonist to a tumor, including the tumor vasculature.

The terms “targeting” and “homing”, as used herein to describe the in vivo activity of a ligand following administration to a subject, each refer to the preferential movement and/or accumulation of a ligand in a target tissue (e.g., a tumor) as compared with a control tissue.

The term “control tissue” as used herein refers to a site suspected to substantially lack binding and/or accumulation of an administered ligand. For example, in one embodiment, a non-cancerous tissue can be a control tissue.

The terms “selective targeting” of “selective homing” as used herein each refer to a preferential localization of a ligand that results in one embodiment in an amount of ligand in a target tissue that is about 2-fold greater than an amount of ligand in a control tissue, in another embodiment in an amount that is about 5-fold or greater, and in still another embodiment in an amount that is about 10-fold or greater. The terms “selective targeting” and “selective homing” also refer to binding or accumulation of a ligand in a target tissue concomitant with an absence of targeting to a control tissue, or the absence of targeting to all control tissues.

The terms “targeting ligand” and “targeting molecule” as used herein each refer to a ligand that displays targeting activity. In one embodiment, a targeting ligand displays selective targeting. Representative targeting ligands include peptides and antibodies.

The term “peptide” encompasses any of a variety of forms of peptide derivatives, that include amides, conjugates with proteins, cyclized peptides, polymerized peptides, conservatively substituted variants, analogs, fragments, peptoids, chemically modified peptides, and peptide mimetics. Representative peptide ligands that show tumor-binding activity include, for example, those described in U.S. Pat. Nos. 6,180,084 and 6,296,832.

The term “antibody” indicates an immunoglobulin protein, or functional portion thereof, including a polyclonal antibody, a monoclonal antibody, a chimeric antibody, a hybrid antibody, a single chain antibody (e.g., a single chain antibody represented in a phage library), a mutagenized antibody, a humanized antibody, and antibody fragments that comprise an antigen binding site (e.g., Fab and Fv antibody fragments). Representative antibody ligands that can be used in accordance with the methods of the presently claimed subject matter include antibodies that bind the tumor-specific antigens Her2/neu (v-erb-b2 avian erythroblastic leukemia viral oncogene homologue-2; Kirpotin et al., 1997; Becerril et al., 1999) and antibodies that bind to CEA (carcinoembryonic antigen; Ito et al., 1991). See also U.S. Pat. Nos. 5,111,867; 5,632,991; 5,849,877; 5,948,647; 6,054,561 and PCT International Publication No. WO 98/10795.

In an effort to identify ligands that are capable of targeting to multiple tumor types, targeting ligands have been developed that bind to target molecules present on tumor vasculature (Baillie et al., 1995; Pasqualini & Ruoslahti, 1996; Arap et al., 1998; Burg et al., 1999; Ellerby et al., 1999).

A targeting ligand can also comprise a ligand that specifically binds to a radiation induced target molecule. Ionizing radiation induces proteins in tumor vascular endothelium through transcriptional induction and/or posttranslational modification of cell adhesion molecules such as integrins (Hallahan et al., 1995; Hallahan et al., 1996; Hallahan et al., 1998; Hallahan & Virudachalam, 1999). For example, radiation induces activation of the integrin α_{2b}β₃, also called the fibrinogen receptor, on platelets. The induced molecules can serve as binding sites for targeting ligands. A representative peptide ligand that binds to irradiated tumors comprises Biapcitide (ACUTECT® available from Diatide, Inc. of Londonberry, N.H., United States of America), which specifically binds to glycoprotein (GP) IIb/IIIa receptors on activated platelets (Hawiger et al., 1989; Hawiger & Timmons, 1992; Hallahan et al., 2001).

Antibodies, peptides, or other ligands can be coupled to drugs (e.g., a PI3K antagonist) or drug carriers using methods known in the art, including but not limited to carbodiimide conjugation, esterification, sodium periodate oxidation followed by reductive alkylation, and glutaraldehyde crosslinking. See e.g., Bauminger & Wilchek, 1980; Dracopoli et al., 1997; Goldman et al., 1997; Kirpotin et al., 1997; Neri et al., 1997; Park et al., 1997; Pasqualini et al., 1997; U.S. Pat. No. 6,071,890; and European Patent No. 0 439 095. Alternatively, pseudotyping of a retrovirus can be used to target a virus towards a particular cell (Marin et al., 1997).

A composition of the presently claimed subject matter comprises in one embodiment a PI3K antagonist and a pharmaceutically acceptable carrier. Suitable formulations include aqueous and non-aqueous sterile injection solutions, which can contain anti-oxidants, buffers, bacteriostats, bactericidal antibiotics, and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Examples of useful ingredients are sodium dodecyl sulfate (SDS), for example in the range of 0.1 to 10 mg/ml, in another example about 2.0 mg/ml; and/or mannitol or another sugar, for example in the range of 10 to 100 mg/ml, in another example about 30 mg/ml; phosphate buffered saline (PBS), and any other formulation agents conventional in the art.

The therapeutic regimens and pharmaceutical compositions of the presently claimed subject matter can be used with additional adjuvants or biological response modifiers including, but not limited to, the cytokines interferon alpha (IFN-α), interferon gamma (IFN-γ), interleukin 2 (IL2), interleukin 4 (IL4), interleukin 6 (IL6), tumor necrosis factor (TNF), or other cytokine affecting immune cells.

IV. Therapeutic Methods

The disclosed PI3K antagonists are useful as radiosensitizing agents. Thus, in one embodiment, the presently claimed subject matter provides a method for suppressing tumor growth comprising: (a) administering a PI3K antagonist to a subject bearing a tumor to increase the radiosensitivity of the tumor; and (b) treating the tumor with ionizing radiation, whereby tumor growth is delayed. Also provided is a method for inhibiting tumor blood vessel growth via administration of a PI3K antagonist.

While applicants do not intend to be bound by any particular theory of operation, a PI3K antagonist is believed to effectively suppress tumor growth by blocking reperfusion of an irradiated tumor. Specifically, a PI3K antagonist can block processes that require PI3K, including the mediation of growth factor signals that result in endothelial cell infiltration and budding of tumor blood vessels. Similarly, a PI3K antagonist is believed to effectively inhibit the growth of tumor blood vessels by blocking the ability of growth factors to mediate blood vessel growth. For example, a PI3K antagonist can block processes mediated by VEGF signal transduction.

IV.A. Administration of a PI3K Antagonist

Suitable methods for administration of a composition of the presently claimed subject matter include but are not limited to intravascular, subcutaneous, intramuscular, intraperitoneal, or intratumoral administration. For delivery of compositions to pulmonary pathways, compositions can be administered as an aerosol or coarse spray. A delivery method is selected based on considerations such as the type of PI3K antagonist, the type of carrier or vector, toxicity of the PI3K antagonist, therapeutic efficacy of the PI3K antagonist, and the condition of the tumor to be treated. In one embodiment of the presently claimed subject matter, intravascular administration is employed.

In one embodiment, a therapeutically effective amount of a composition of the presently claimed subject matter is administered to a subject. A “therapeutically effective amount” is an amount of a composition comprising a PI3K antagonist sufficient to produce a measurable anti-tumor response (e.g., an anti-angiogenic response, a cytotoxic response, and/or tumor regression). Actual dosage levels of active ingredients in a therapeutic composition of the presently claimed subject matter can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, tumor size and longevity, and the physical condition and prior medical history of the subject being treated. Determination and adjustment of a therapeutically effective dose, as well as evaluation of when and how to make such adjustments, are known to those of ordinary skill in the art of medicine.

In one embodiment of the presently claimed subject matter, a minimally therapeutic dose of a PI3K antagonist is administered. The term “minimally therapeutic dose” refers to the smallest dose, or smallest range of doses, determined to be a therapeutically effective amount as defined herein above.

IV.B. Radiation Treatment

For treatment of a radiosensitized target tissue, the target tissue is irradiated concurrent with, or subsequent to, administration of a composition comprising a PI3K antagonist. In accordance with the methods of the presently claimed subject matter, the target tissue can be irradiated daily for 2 weeks to 7 weeks (for a total of 10 treatments to 35 treatments). Alternatively, target tissues can be irradiated with brachytherapy utilizing high dose rate or low dose rate brachytherapy internal emitters.

Subtherapeutic or therapeutic doses of radiation can be used for treatment of a radiosensitized target tissue as disclosed herein. In one embodiment, a subtherapeutic or minimally therapeutic dose (when administered alone) of ionizing radiation is used. For example, the dose of radiation can comprise at least about 2 Gy ionizing radiation, in another example about 2 Gy to about 6 Gy ionizing radiation, and in yet another example about 2 Gy to about 3 Gy ionizing radiation. When radiosurgery is used, representative doses of radiation include about 10 Gy to about 20 Gy administered as a single dose during radiosurgery or about 7 Gy administered daily for 3 days (about 21 Gy total). When high dose rate brachytherapy is used, a representative radiation dose comprises about 7 Gy daily for 3 days (about 21 Gy total). For low dose rate brachytherapy, radiation doses typically comprise about 12 Gy administered twice over the course of 1 month. ¹²⁵I seeds can be implanted into a target tissue and can be used to deliver very high doses of about 110 Gy to about 140 Gy in a single administration.

Radiation can be localized to a target tissue using conformal irradiation, brachytherapy, stereotactic irradiation, or intensity modulated radiation therapy (IMRT). The threshold dose for treatment can thereby be exceeded in the target tissue but avoided in surrounding normal tissues. For treatment of a subject having two or more target tissues, local irradiation enables differential drug administration and/or radiotherapy at each of the two or more target tissues. Alternatively, whole body irradiation can be used, as permitted by the low doses of radiation required following radiosensitization of the target tissue.

Radiation can also comprise administration of internal emitters, for example ¹³¹I for treatment of thyroid cancer, NETASTRON™ and QUADRAGEN® pharmaceutical compositions (Cytogen Corp. of Princeton, N.J., United States of America) for treatment of bone metastases, and ³²P for treatment of ovarian cancer. Other internal emitters include ¹²⁵I, iridium, and cesium. Internal emitters can be encapsulated for administration or can be loaded into a brachytherapy device.

Radiotherapy methods suitable for use in the practice of this presently claimed subject Matter can be found in Leibel & Phillips, 1998, among other sources.

### EXAMPLES

The following Examples have been included to illustrate modes of the presently claimed subject matter. Certain aspects of the following Examples are described in terms of techniques and procedures found or contemplated by the present inventor to work well in the practice of the presently claimed subject matter. These Examples illustrate standard laboratory practices of the inventors. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1

### Akt is Phosphorylated in Response to Irradiation

HUVEC cells were obtained from Clonetics and were maintained in EBM-2 medium supplemented with EGM™-2MV SINGLEQUOTS® (Bio-Whittaker, Inc., Walkersville, Md., United States of America). An Eldorado-8 Cobalt-60 teletherapy unit (Atomic Energy of Canada Limited, Mississauga, Ontario, Canada) was used to irradiate endothelial cell cultures at a dose rate of 0.84 Gy per minute. Delivered doses ranging from 0 to 12 Gy were verified by use of thermoluminescence detectors.

In order to determine if Akt is phosphorylated in response to irradiation, cultured HUVEC cells were serum starved for 4 hours, exposed to irradiation, and the phosphorylation of Akt was examined in cell extracts by Western blotting. Total cellular protein was extracted after radiation treatment as follows. Cells were counted and then washed with iced-cold PBS twice before the addition of lysis buffer (M-PER™ mammalian protein extraction reagent; Pierce Chemical Company, Rockford, Ill., United States of America; or 20 mM Tris, 150 mM NaCl, 1 mM EDTA, 1% Triton X-100, 2.5 mM sodium pyrophosphate, 1 mM phenylmethylsulphonyl fluoride (PMSF), and 1 mg/ml Leupeptin (Roche Molecular Biochemicals, Indianapolis, Ind., United States of America)). Protein concentrations were quantified using the Biorad method (Bradford, 1976). 50 μg protein was loaded into each well of an 8% sodium dodecyl sulfide-polyacrylamide gel electrophoresis (SDS-PAGE) gel and separated by electrophoresis. The proteins were subsequently transferred onto 0.45 μm nitrocellulose membranes. Membranes were blocked by incubation for 2 hours at room temperature in phosphate buffered saline-Triton (PBST: 0.9% NaCl, 0.3% Triton X-100, pH 7.4) containing 10% nonfat dry milk.

The presence of Akt protein on the membrane was determined as follows. The membranes were incubated with a 1:1000 dilution of rabbit antibody directed against either phosphorylated (Ser473) or non-phosphorylated human Akt overnight at 4° C. Rabbit antibodies were obtained from Cell Signaling Technology, Beverly, Mass., United States of America. The blots were then washed with PBST to remove unbound rabbit antibodies. The washed blots were then incubated for 1 hour at room temperature with a donkey anti-rabbit IgG secondary antibody (Amersham Biosciences, Piscataway, N.J., United States of America) diluted 1:1000 in PBST. The blots were again washed with PBST to remove unbound donkey antibodies. The presences of both phosphorylated and non-phosphorylated human Akt were determined by chemiluminescent detection of donkey anti-rabbit/rabbit anti-human Akt complexes using the ECL™ detection system (Amersham Biosciences) according to the manufacturer's protocol, followed by autoradiography.

Dose-dependent phosphorylation of Akt occurred rapidly after treatment with radiation. A radiation dose of 1 Gy led to an increase in Akt phosphorylation, with doses of 3, 6, 9, and 12 Gy resulting in considerable Akt phosphorylation. These effects were not due to the induction of additional Akt protein synthesis, as the amount of Akt present in the cells did not change detectibly following irradiation.

Phosphorylation of Akt was also time-dependent. A radiation dose of 3 Gy induced Akt phosphorylation within 5 minutes and peak Akt phosphorylation occurred 15 minutes after irradiation. Akt phosphorylation was maintained at 30 minutes, but by 60 minutes the phosphorylated Akt content of the cell returned to pre-irradiation levels. This effect was also not due to the induction of additional Akt protein synthesis, as the amount of Akt present in the cells did not change detectibly following irradiation.

### Example 2

### Akt Phosphorylation in Response to Irradiation is Mediated by PI3K

Wortmannin (Sigma-Aldrich Corp., St. Louis, Mo., United States of America) was stored in the dark at 4° C. and dissolved in dimethylsulfoxide (DMSO). LY294002 [2-(4-Morpholinyl)-8-phenyl-4H-1-benzopyran-4-one; BIOMOL Research Laboratories, Inc., Plymouth Meeting, Pa., United States of America] was stored in DMSO at −20° C. Wortmannin and/or LY294002 were diluted in medium immediately before administration to cell cultures.

In order to test whether or not the phosphorylation of Akt in response to irradiation is mediated by PI3K, HUVEC cells were exposed to radiation as described in Example 1 either with or without prior treatment with various PI3K antagonists. For those cultures treated with antagonist, either wortmannin (4 nM) or LY294002 (2 μM) were added to cultures 30 minutes prior to irradiation. Controls used included untreated cells, cells treated with one antagonist or the other alone, and radiation alone. As a positive control for Akt phosphorylation, HUVEC cells treated with vascular endothelial growth factor (VEGF) were also examined since exposure of HUVEC cells to VEGF has been shown to result in rapid phosphorylation of Akt.

After radiation treatment, total cellular protein was isolated, separated by SDS-PAGE, and transferred to nitrocellulose filters as described in Example 1. Total Akt protein levels in each lane were assayed by Western analysis as described in Example 1.

The phosphorylation of Akt is mediated by PI3K as evidenced by the strong inhibition of Akt phosphorylation when either wortmannin (4 nM) or LY294002 (2 μM) were added to the HUVEC cultures 30 minutes prior to irradiation. Either wortmannin or LY294002 inhibited Akt phosphorylation following treatment of HUVEC with 3 Gy. These PI3K antagonists had no effect on total Akt levels.

Wortmannin and LY294002 have been shown to inhibit DNA-PK activity (Rosenzweig et al., 1997), so in order to determine if the inhibition of Akt phosphorylation attributed to wortmannin and LY294002 was due to specific inhibition of PI3K activity, a dominant negative PI3K construct was introduced into HUVEC cells. Adenovirus vector AxCAΔp85 (Sakaue et al., 1997; Kitamura et al., 1998) was obtained from Wataru Ogawa (Kobe University School of Medicine, Kobe, Japan). It encodes a mutant p85 regulatory subunit that is unable to bind the p110 subunit of PI3K (Hara et al., 1994). HUVEC cells were transduced with AxCAΔp85 or a similar recombinant adenovirus encoding green fluorescent protein (GFP) at 10-100 plaque-forming units (pfu)/cell. 24 hours post-infection, infected cells were subjected to treatment with irradiation and analyzed as described in Example 1. Briefly, adenovirus-containing cells were treated with 3 Gy irradiation and total cellular protein was isolated a 0, 15, 30, and 60 minutes post-irradiation. Isolated proteins were analyzed by immunoblot and the phosphorylation of Akt at these time points was determined. The GFP-encoding adenovirus vector did not affect the phosphorylation of Akt, as Akt phosphorylation in these cells mirrored that seen in HUVEC cells that did not harbor an adenovirus vector. However, HUVEC cells containing the AxCAΔp85 vector showed undetectable Akt phosphorylation at 15 minutes, and barely detectible phosphorylation at 30 minutes. Thus, the dominant negative PI3K construct virtually eliminated Akt phosphorylation, indicating that the inhibition of radiation-induced Akt phosphorylation observed when HUVEC cells are pre-treated with wortmannin or LY294002 is mediated by PI3K.

### Example 3

### PI3K Antagonists Enhance Apoptosis in Response to Ionizing Radiation

To determine whether PI3K antagonists enhance the activities of intrinsic cell death mechanisms induced by irradiation, apoptosis in HUVEC cells was studied following PI3K inhibition. HUVEC cells were treated with wortmannin (4 nM) or with LY294002 (2 μM) as described in Example 2. Cells were treated 30 minutes later with 3 Gy of ionizing radiation. Cultures were stained 24 hours later with hematoxylin and eosin (H&E) to reveal apoptotic nuclei. Stained cells were then examined by light microscopy. For each treatment group, four high power fields (40× objective) were examined, and both the number of cells with apoptotic nuclei and the total number of cells were determined. From these numbers, the percentage of apoptotic cells for each group was calculated.

Only 2% of cells were undergoing apoptosis in the population of control cells not exposed to ionizing radiation. Unirradiated cells exposed to wortmannin or to LY294002 showed 3% and 2% apoptotic cells, respectively (p=0.8). Radiation treatment alone induced a moderate increase in apoptosis (5-6% of cells; p<0.05, Bonferroni t test). However, pretreatment of HUVEC cells with wortmannin or LY294002 significantly enhanced radiation-induced apoptosis. HUVEC cells pretreated with LY294002 or wortmannin prior to irradiation showed an increase in apoptotic nuclei to 16% and 17% of cells, respectively (p<0.001).

Apoptosis was confirmed by the presence of cellular DNA fragmentation. DNA fragmentation was assayed as follows. After treatment with radiation and/or PI3K antagonists, HUVEC cells were placed in the incubator for 24 hours. The medium was collected in centrifuge tubes to retain any floating, apoptotic cells. The remaining cells were trypsinized and added to the collected medium. The tubes were spun down at 2500 rpm for 10 minutes at 4° C. To the cell pellets, 500 μL of DNA Lysis Buffer (5 mM Tris-HCl pH 7.4, 20 mM EDTA, and 0.5% Triton X-100) was added, along with 25 μL of 20 mg/mL proteinase K and 60 μL of 10% SDS. This mixture was incubated at 50° C. for 1 hour. 600 μL of chloroform was then added. The tubes were shaken and then spun for ten minutes at 14,000 RPM. The aqueous layer was removed to a new tube and 2 volumes of cold 95% ethanol were added. The DNA was allowed to precipitate for one hour followed by centrifugation as before. The DNA pellet was then resuspended in TE (10 mM Tris, 1 mM EDTA, pH 8.0). 10 μg of DNA was run on a 2.0% agarose gel stained with ethidium bromide. The gel was then photographed under UV light. DNA laddering typical of apoptosis was observed.

### Example 4

### PI3K Antagonists Suppress Clonogenic Potential in Response to Radiation

The ability of PI3K antagonist to suppress the clonogenic potential of HUVEC cells treated with radiation was assayed by clonogenic survival analysis. The ability of HUVEC cells treated with LY294002 30 minutes prior to irradiation to form colonies was compared to that of HUVEC cells treated with either radiation or LY294002 alone, and also to that of untreated HUVEC cells. Clonogenic survival analysis was performed as previously described (Geng et al., 2001). Briefly, groups of three HUVEC culture plates were treated at each of four radiation dose levels: 2, 4, 6, and 8 Gy, either with or without prior treatment with PI3K antagonists as described in Example 2. After treatment with radiation and/or antagonists, cells were trypsinized, counted by hemocytometer, and subcultured into fresh medium. After 14 days, the cells were fixed with cold methanol and stained with 1% methylene blue. Colonies with greater than 50 cells were counted and the surviving fraction was determined. The plating efficiency of unirradiated HUVEC cells treated with LY294002 for 30 minutes was reduced to 70% of that seen for untreated control cells. LY294002 treatment significantly enhanced radiation-induced cytotoxicity compared to HUVEC treated with radiation alone (p<0.05, Bonferroni t test).

### Example 5

### Tumor Models

The GL261 glioblastoma cell line was obtained from Dr. Daryl Bigner (Duke University, Durham, N.C., United States of America). GL261 cells were maintained in Dulbecco's Modified Eagle Medium (DMEM) with Nutrient Mixture F-12 1:1 (Invitrogen Corporation, Carlsbad, Calif., United States of America) with 7% fetal calf serum, 0.5% penicillin/streptomycin, and 1% sodium pyruvate. Cells were incubated in a 37° C., 5% CO₂ incubator. GL261 cells form tumors following injection into a dorsal skin fold window chamber as described in Example 6 or subcutaneous injection into the hind limbs of C57BL/6J mice as described in Example 7. Additionally, the GL261 glioblastoma model shows high levels of VEGF expression and radiation resistance in vivo (Gorski et al., 1999). Prior to implantation in the mouse, cells were trypsinized and counted using a hemocytometer. Cells were washed in complete medium and 10⁶ cells were injected subcutaneously into the hind limb or into the dorsal skin fold window as described in the Examples below.

### Example 6

### Tumor Vascular Window Model

Penicillin/streptomycin solution (200 μl; available from GIBCO™ Invitrogen Corporation of Carlsbad, Calif., United States of America) was injected into the hind limb of the mouse prior to preparation of the window chamber. The dorsal skin fold window chamber (Leunig et al., 1992) was prepared using a 3-gram plastic frame, which was applied to the skin of a test animal and remained attached for the duration of the study.

To prepare a test animal for application of the plastic frame, the dorsal midline was found along the animal's back, and a clip was placed to hold the skin in position. A template, equivalent to the outer diameter of the chamber, was traced, producing the outline of the incision. A circular cut was made tracing the perimeter of the outline (a circular area about 7 mm in diameter), followed by a crisscross cut through the center of the circular area, thus producing four skin flaps. The epidermis of the four flaps was removed using a scalpel with an effort to follow the hypodermis superior to the fascia. The area was then trimmed and manicured with a pair of fine forceps and iris scissors. During surgery, the area was kept moist by applying moist drops of phosphate buffered saline (PBS) with 1% penicillin/streptomycin solution.

The template was removed and the top piece of the chamber was fixed with screws. The bottom portion of the chamber was put in place and the top portion of the chamber was carefully positioned on the cut side, so that the window and the circular incision were fitted. Antibiotic ointment was applied to keep the area clear of infection. The three screws that hold the chamber together were then put into the chamber holes and tightened so that the skin was not pinched, thus avoiding diminished circulation.

10⁶ GL261 cells were implanted into the dorsal skin fold window chamber. Blood vessels developed over 1 week. Tumor vasculature was observed in response to various treatments as described below. To document changes in tumor blood vessels, the window frame was marked with coordinates, which were used to photograph the same microscopic field prior to treatment (0 hours) and at 96 hours post-treatment. Vascular windows were photographed using a 4× objective to obtain a 40× total magnification. Color slides were scanned into PHOTOSHOP® software (Adobe Systems, Inc., San Jose, Calif., United States of America). Scanned slides were analyzed using IMAGE-PRO® software (Media Cybernetics, Silver Spring, Md., United States of America) as described below.

Vascular centerlines were positioned by IMAGE-PRO® software and verified by an observer blinded to the treatment groups. Tumor blood vessels were quantified using IMAGE-PRO® software, which quantified the vascular length density of blood vessel within the microscopic field. The mean vascular length density and the standard error of the mean were calculated for each treatment group. The mean and 95% confidence intervals of vascular length density for each treatment group were calculated and variance was analyzed by the General Linear Models and Bonferroni t test.

The vascular window model allowed direct measurement of the vascular response to ionizing radiation. Five mice were studied in each of the treatment groups. LY294002 (3 mg/Kg) was injected intraperitoneally 15 minutes prior to irradiation into appropriate treatment groups. Vascular windows were treated with 3 Gy superficial X-rays using 80 kVp (Pantak X-ray Generator). The mean vascular length densities of treatment group were determined at 0 hours, which was the time at which the radiation treatments were commenced. For each treatment group, the mean vascular length densities at 0 hours were defined as 100%.

At 96 hours post-irradiation, the mean vascular length densities were determined for each treatment group and compared to the mean vascular length densities of each treatment group at 0 hours. The mean vascular length density of tumor blood vessels receiving neither radiation nor antagonist treatment increased to 111% as compared to the same vessels at the 0 hour time point. Administration of LY294002 in the absence of radiation treatment achieved insignificant regression of tumor blood vessels (85%; p=0.41). Tumors treated with 3 Gy alone showed minimal vascular response to radiation (63%; p=0.13). However, treatment with LY294002 prior to irradiation resulted in a marked reduction in mean vascular length density at 96 hours, which was 5% of that seen at the 0 hour time point.

### Example 7

### Power Doppler Sonography

Power Doppler sonography was used to study the response of tumor blood vessels to ionizing radiation. This method utilizes amplitude to measure blood flow in microvasculature. Tumors were imaged with a 10-5 MHz ENTOS® linear probe (Advanced Technology Laboratories, Inc., Bothell, Wash., United States of America) attached to an HDI® 5000 diagnostic ultrasound system (Advanced Technology Laboratories, Inc.). Power Doppler sonography images were obtained with the power gain set to 82%. Care was taken to minimize motion artifact. A 20-frame cineloop sweep of the entire tumor was obtained with the probe perpendicular to the long axis of the lower extremity along the entire length of the tumor.

The images were analyzed using HDI® software (Advanced Technology Laboratories, Inc). This software allows direct evaluation of Power Doppler cineloop raw data. Color area was recorded for the entire tumor. Five mice were included in each treatment group. Values for color area were averaged for each tumor set and treated groups were compared to controls using the unpaired student t-test.

GL261 glioma tumors were implanted in the hind limb of C57BL/6J mice as described in Hallahan et al., 1998. Briefly, 10⁶ viable GL261 cells suspended in 0.2 ml of a 0.6% agarose solution were introduced into the hind limb of the mouse by subcutaneous injection. Tumors were grown to a diameter of about 0.7 cm to about 1.1 cm and then irradiated with 3 Gy ionizing radiation. Power Doppler analysis of tumor blood flow was measured on day 0, day 3, and day 7 after radiation treatment. Doppler sonography showed that tumor blood flow was obliterated in response to treatment with LY294002 prior to irradiation. Blood flow was unchanged 20′ after treatment with either radiation or LY294002 alone.

### Example 8

### Tumor Volume Assessment

GL261 tumors were generated by subcutaneous injection of 10⁶ cells into the right thighs of C57BL/6 mice as described in Example 6. Mice were stratified into four groups so that the mean tumor volume of each group was comparable. An equal number of large and intermediate size tumors were present in each group.

GL261 tumor-bearing mice were treated with 24 Gy ionizing radiation administered in 8 fractionated doses over 10 days. LY294002 was administered at 3 mg/kg by intraperitoneal injection 15 minutes prior to irradiation on days 1, 3, 5, and 7 of radiation therapy. Control mice received identical doses and schedules of either LY294002 alone or radiation alone.

Twice each week, tumor volumes were measured using skin calipers as previously described (Grugel et al., 1995; Advani et al., 1998; Staba et al., 1998). Briefly, tumor volumes were calculated using the formula:
a×b×c/2,
where a=maximal width dimension, b=maximal length dimension, and c=maximal depth dimension, which was derived from the formula for calculating the volume of an ellipsoid (πd³/6). Data were calculated as the percent of original (day 0) tumor volume and graphed as fractional tumor volume plus or minus one standard deviation for each treatment group.

Growth delay was defined as the number of days required for tumors to reach a volume of 1 cm³. Untreated control tumors reached 1 cm³ at 7 days. Tumors treated with either radiation or LY294002 alone showed minimal growth delay compared to untreated controls (9 days). However, the combination of LY294002 followed by radiation significantly delayed tumor growth when compared to radiation or LY294002 alone (p=0.04). Tumors in mice treated with LY294002 prior to irradiation demonstrated growth delay that extended beyond 15 days.

### Example 9

### Antibodies and Other Reagents

An adenovirus encoding a mutant regulatory subunit of p85 (Ad.Δp85) was kindly provided by Wataru Ogawa (Kobe University School of Medicine, Kobe, Japan; see Kotani et al., 1999; Sakaue et al., 1997). Antibodies to phosphoserine-473 Akt, total Akt, caspase 3, caspase 9, and caspase 9 cleaved fragment were obtained from New England Biolabs (Beverly, Mass., United States of America). Antibodies to cytochrome C and anti-poly (ADP-ribose)polymerase (PARP) were obtained from Oncogene Research Products (Boston, Mass., United States of America). Antibodies to cytochrome C oxidase subunit IV (CytOx4) were obtained from CLONTECH™ (Palo Alto, Calif., United States of America). Antibodies to actin and tubulin were obtained from Santa Cruz Biotechnology Inc. (Santa Cruz, Calif., United States of America).

### Example 10

### Caspase and PARP Assays

Cells were also assayed for apoptosis, caspase 3, and PARP cleavage by Western immunoblot. Apoptosis assays utilized propidium iodide staining of nuclei. Apoptotic nuclei were counted, and the percentage of cells undergoing apoptosis was quantified in three separate experiments. The mean and standard of the mean were calculated. Caspase 3 inhibitor IV was obtained from Oncogene Research Products, (Boston, Mass., United States of America). Caspase inhibitor was added to cells at a concentration of 100 μM for 60 minutes prior to irradiation. Statistical differences of p<0.05 using the Student's t test were considered significant.

### Example 11

### Cell Lysis and Immunoblot Analysis

HUVEC were treated at the indicated times and washed twice with PBS and lysed in lysis buffer (20 nM Tris, 150 mM NaCl, 1 mM EDTA, 1% Triton X-100, 2.5 mM sodium pyrophosphate, 1 mM phenylmethylsulfonyl fluoride, and 1 μg/ml leupeptin). Protein concentrations were quantified by the BioRad method. 20 μg of total protein were loaded into each well and separated by 8 or 12% SDS-PAGE gel, depending on the size of the target protein being investigated. The proteins were transferred onto nitrocellulose membranes (Hybond ECL, Amersham, Arlington Heights, Ill., United States of America) and probed with antibodies for the phosphorylated Akt, total Akt, caspase 3, caspase 9, cytochrome C, or PARP.

### Example 12

### Irradiation and Cytochrome Release

Apoptosis is preceded by cytochrome C release from mitochondria (Green & Reed, 1998). To determine whether cytochrome C is released from the mitochondria following irradiation, the subcellular localization of cytochrome C was assessed. Cells were lysed in hypotonic fractionation buffer mix from CLONTECH™ (Palo Alto, Calif., United States of America), incubated on the ice for 10 minutes, then homogenized in an ice-cold Dounce tissue grinder from Fisher Scientific (St. Louis, Mo., United States of America). Cell homogenates were transferred to 1.5 ml microcentrifuge tubes, and centrifuged at 700×g for 10 minutes. Proteins within the supernatant were cytosolic fractions, with the pellet containing mitochondrial fractions. 5-10 μg of each cytosolic and mitochondrial fraction isolated from induced cells and control cells were loaded onto a 12% SDS-PAGE gel. After separation, proteins were transferred to nitrocellulose, which were probed with an anti-cytochrome C antibody at a 1:100 dilution in blocking buffer.

Autoradiographs of Western immunoblots using antibody specific for cytochrome C were prepared. Immunoblots using antibodies specific for cytochrome C oxidase 4 and actin were used as controls. Western blot analysis revealed that mitochondrial cytochrome C decreased, while cytochrome C in the cytosolic fraction increased in HUVEC treated with radiation and Ad.Δp85. Cytochrome C remained within the mitochondria following treatment with either Ad.Δp85 alone or radiation alone.

### Example 13

### Involvement of Caspases 3 and 9 in Akt-Mediated Apoptosis

Cytochrome C activates the apoptotic cascade through cleavage of caspase 9. To characterize the mechanisms by which Ad.Δp85 regulates apoptosis in irradiated endothelial cells, caspases 3 and 9 were studied. HUVEC cells were transduced with Ad.GFP or Ad.Δp85 for 24 hours followed by irradiation. Total protein was extracted at 6 hours after irradiation. Autoradiographs of Western immunoblot using antibody specific for caspases 3 and 9 were prepared. Following serum starvation, each of the caspases was cleaved to form proteolytic products. Caspases 3 and 9 were not cleaved following treatment with either Ad.Δp85 alone or radiation alone. HUVEC transduced with Ad.Δp85 showed cleavage of caspases 3 and 9 at 6 hours following irradiation.

Upon activation, caspase 3 cleaves numerous proteins involved in cell structure, signaling, and repair, and is essential for DNA fragmentation. PARP is one of the downstream substrates for caspase 3 (Granville et al., 1998). To determine whether caspase 3 activation is essential for apoptosis induced by radiation and mutant Ad.Δp85, HUVEC were treated with caspase 3 inhibitor IV. PARP cleavage and subsequent apoptosis were studied in Western immunoblots using anti-PARP antibodies. Following treatment with radiation and Ad.Δp85, PARP was cleaved within endothelial cells, whereas caspase 3 inhibitor IV prevented PARP cleavage. Caspase 3 inhibitor IV also prevented apoptosis induced by radiation in Ad.Δp85 transduced endothelial cells. Untreated control and Ad.GFP transduced cells each had 3% of apoptotic cells. Cells treated with 3 Gy alone or Ad.GFP+3 Gy had 6%, and 7% apoptotic cells, respectively. Cells treated with Ad.Δp85 and 3 Gy showed 21% apoptotic cells. Caspase inhibitor IV added to cells treated with Ad.Δp85 and 3 Gy reduced the percentage of apoptotic cells to 3%.

### Discussion of Examples 9-13

Akt attenuation of programmed cell death occurs through several independent mechanisms. One such mechanism involves the Bcl gene family members, which are directly phosphorylated by Akt and participate in the Akt-mediated anti-apoptotic effect (Krasilnikov, 2000). Bcl-2 and p53 are two well-characterized proteins known to regulate radiation-mediated apoptosis. Bcl-2 and its homologue, Bcl-x encode membrane-associated proteins that protect cells from DNA damage-induced apoptosis.

As disclosed herein, PI3K inhibitor or radiation alone can promote HUVEC apoptosis. Most importantly, the number of apoptotic bodies within HUVEC increased dramatically when treated by the PI3K inhibitor and radiation together as compared to treatment with radiation or the PI3K dominant negative alone. Additionally, a radiation dose- and time-dependent activation of Akt phosphorylation in endothelial cells was found. PI3K inhibition resulted in enhancement of endothelial apoptosis and diminished viability, suggesting that the PI3K participates in radiation-induced phosphorylation of Akt and subsequent enhancement of cell viability.

Akt-enhanced cell survival might occur through maintenance of mitochondrial integrity. Induction of apoptosis by Akt inactivation correlated with the disruption of mitochondrial membrane integrity and cytochrome c release. Radiation might induce the loss of mitochondrial membrane potential, opening of the permeability transition pore and the release of cytochrome C (Taneja et al., 2001). The present disclosure shows that PI3K dominant negative promoted apoptosis within irradiated HUVEC. Cytochrome C was released into the cytosol only upon treatment with radiation and mutant p85 together. Cytochrome C release into cytosol interacts directly with Apaf-1 in the cytoplasm leading to the ATP dependent formation of the apoptosome. This complex recruits and activates the protease caspase 9 (Brunelle & Chandel, 2002). Caspase 3 has been shown to be indispensable for apoptosis induced by numerous cytotoxic stimuli (Janecki et al., 2000). For that reason, caspase 3 inhibitor IV was used to determine whether caspase 3 is essential for apoptosis induced within HUVEC treated with Ad.Δp85 and radiation.

The resilience of vascular endothelium to cytotoxic effects of ionizing radiation can be overcome by overexpression of p85 with mutation within the SH2 domain. This genetic construct inhibited radiation-induced Akt phosphorylation and subsequently enhances radiation-induced apoptosis within vascular endothelial cells. These findings support PI3K as a therapeutic target in aberrant vascular endothelium during treatment with ionizing radiation.

### Example 14

### Effect of SU11248 on Radioresistance

To determine whether prolonged growth delay correlated with reduction in tumor blood flow, amplitude modulated Power Doppler was used to monitor blood flow. Reduced blood flow in tumors treated with SU11248 and radiation correlated with improved tumor growth delay. Tumors treated with SU11248 and radiation approached significant reduction in blood flow as compared to tumors treated with radiation alone (p<0.053).

To determine whether broad spectrum RTK inhibition enhances the cytotoxic effects of radiation on vascular endothelium, SU11248 was added 30 minutes prior to irradiation. HUVECs were treated with vehicle control, and either 100 nM SU11248 orvehicle combined with 6 Gy. The percentage of endothelial cells demonstrating apoptotic nuclei 24 hours post treatment was determined for each experimental group. Untreated control cells show 2% apoptotic nuclei as compared to 7 and 8% after treatment with SU11248 or radiation, respectively (p>0.1). HUVEC treated with SU11248 followed by 6 Gy showed 21% of cells with apoptotic nuclei at 24 hours, which was significantly greater than either agent alone (p<0.02), or untreated control cells (p<0.001). To determine whether enhanced apoptotic response in endothelial cells treated with SU11248 results in reduced clonogenic cell survival, HUVEC were subcultured and colony formation was quantified. HUVEC treated with SU11248 prior to irradiation showed a significant reduction in clonogenic survival as compared to radiation alone (p<0.05).

Growth factors produced by tumors could enhance the viability of tumor vascular endothelium. To determine whether SU11248 enhances radiation-induced destruction of tumor vasculature, SU11248 was administered to mice prior to irradiation with 3 Gy. Tumor vascular linear density was measured by use of intravital tumor vascular window. Representative photographs of tumor vasculature before and 48 hours after treatment with SU11248, 3 Gy, or SU11248 followed by 3 Gy indicated that RTK inhibition increases tumor vascular destruction as compared to either agent alone. Five mice were treated in each of the treatment groups, and the vascular length density after treatment was quantified. Mean vascular length densities over four days are shown as a bar graph. Within 72 hours, vascular length density in tumors was significantly reduced to 8% of that at 0 hours (p<0.01). In comparison, tumors treated with either 3 Gy or SU11248 alone showed an insignificant reduction in vascular length density to 75 and 84% that of 0 hour, respectively. Combined SU11248 and 3 GY achieved significantly greater reduction in vascular length density as compared to either agent alone (p<0.05).

Destruction of tumor vasculature results in ischemia, which could reduce radiosensitivity of tumors. To determine whether SU11248 enhances tumor growth delay in irradiated tumors, mice bearing LLC and GL261 hind limb tumors were treated with intraperitoneal injection of 40 mg/kg SU11248 or control vehicle 30 minutes prior to each 3 Gy dose of radiation for a total of seven administrations. Both the inhibitor and radiation were discontinued after day 8. Time to doubling of LLC tumor size was 5, 6, 8 and 16 days for vehicle, SU11248, vehicle+21 Gy, and SU11248+21 Gy, respectively. Both LLC and GL261 tumors showed a significant increase in tumor growth delay when SU11248 was added prior to daily 3 Gy fractions as compared to either agent alone (p<0.05).

Upon the discontinuation of therapy, tumors rapidly regrow. To determine whether maintenance of SU11248 beyond the completion of radiation improved growth delay, this RTK inhibitor was administered twice daily continuously in groups treated with radiation alone or SU11248 and radiation. Beginning after day 8, three groups (drug alone, radiation+vehicle, and combined therapy) were maintained with twice daily intraperitoneal administrations of 20 mg/kg SU11248 for an additional 7 days. Maintenance treatment was discontinued after day 17, and subsequently resumed after four days of drug holiday. Each of the maintenance groups showed significantly delays in growth to 4-fold that of day 0 tumors (p<0.05 in all groups). Re-initiation of SU11248 at day 21 produced a second phase of tumor growth delay.

### Example 15

### Effect of SU6668 on Phosphorylation of Akt

Broad spectrum RTK inhibitors were tested for their abilities to attenuate radiation-induced phosphorylation of Akt. SU6668, a tyrosine kinase inhibitor of VEGFR, PDGFR, FGFR, and c-Kit, was added to endothelial cells prior to irradiation. Western immunoblots using phospho-specific antibodies to phosphorylated Akt (P-Akt) were prepared. SU6668 blocked radiation-induced phosphorylation of Akt, whereas SU5416, which is VEGF receptor specific, did not attenuate radiation-induced activation of Akt in endothelial cell cultures.

Phosphorylation of MEK and Erk proteins was also investigated. Erk1/2 was activated following a single 3 Gy dose of irradiation. Erk was phosphorylated within 15 minutes, which resolved within 60 minutes. SU6668 is a broad spectrum inhibitor of split kinase domain RTKs, including Flk-1, Flt-1, PDGFR, fibroblast growth factor receptor (FGFR), and c-Kit (Mendel et al, 2003). SU6668 was added 30 minutes prior to irradiation and attenuated Erk 1/2 phosphorylation, but did not entirely eliminate activation of this pathway.

### Example 16

### PDGF Receptor Activity and Akt Signaling

The role of RTKs in radiation-induced activation of the Akt signaling pathway was examined by studying a panel of tyrosine kinase inhibitors including PDGF receptor antagonists in primary culture endothelial cells. PDGF receptor antagonists including SU6668, SU11248, and STI571, attenuated radiation-induced phosphorylation of both Akt and Erk. Conversely, inhibition of MEKK and ErbB did not inhibit radiation-induced activation of Akt and Erk1/2. Similar efficacy in blocking Erk1/2 phosphorylation in irradiated endothelial cells was found.

To determine to the level of phosphorylation of PDGF receptor, GL261 mouse tumor models were sectioned and stained. A phospho-specific antibody to PDGFRβ Tyr751 (Cell Signaling Technology, Beverly, Mass., United States of America) was used. Tumors produced by injecting GL261 murine glioma cells into the hind limb of C57BL6 mice were stained for phospho-PDGFRβ and visualized using immunohistochemistry. Phospho-PDGFRβ staining was graded as 2+ relative to staining in negative controls.

To determine whether cytotoxic therapy alters PDGFRβ phosphorylation, the tumor was irradiated (3 Gy) and snap frozen following irradiation. Irradiated tumors showed increased phosphorylation of PDGFRβ as compared to untreated controls. Positive staining for phosphorylated PDGFR was found in endothelium and tumor stroma.

To investigate the effect of a PDGF receptor antagonist on phosphorylation, SU11248 was administered one hour prior to 3 Gy, which markedly attenuated radiation-induced PDGFR phosphorylation. Western immunoblots were performed to verify radiation induced an increase in phosphorylation of PDGFRβ. Protein extracted from irradiated HUVEC had increased phosphorylation of PDGFRβ.

### Example 17

### TUNEL Staining of Tumor Sections

To determine whether SU11248 interacts with radiation through the induction of apoptosis in tumor endothelium, TUNEL staining of tumor sections was utilized. Tumor sections were co-stained for the endothelial marker von Willebrand factor (vWF) and TUNEL. Following treatment with either 3 Gy or SU11248 alone, apoptotic cells could not be detected. Tumors treated with radiation and SU11248 showed TUNEL positive endothelial cells with no apoptotic tumor cells at 24 hours following treatment.

### Example 18

### Involvement of SP1 in Radiation-Induced Akt Phosphorylation

Acid sphingomyelinase induces the production of ceramide, which is in turn converted to sphingosine and later S1P. S1P binds to the EDG-1 receptor, which in turn activates PI3K and Akt (Garcia-Barros et al., 2003).

In order to test whether S1P can participate in radiation-induced Akt phosphorylation, two inhibitors of ceramide signal transduction, flumonisin and MAPP, were used. These antagonists were added to HUVEC in culture. HUVEC were irradiated and total protein was extracted. Western immunoblot utilizing the antibody specific for the phosphorylated form of Akt were prepared. Akt was phosphorylated in response to VEGF and 3 Gy irradiation. Flumonisin and MAPP did not attenuate Akt phosphorylation.

### REFERENCES

The references listed below as well as all references cited in the specification are incorporated herein by reference to the extent that they supplement, explain, provide a background for or teach methodology, techniques and/or compositions employed herein.
Advani S J, Sibley G S, Song P Y, Hallahan D E, Kataoka Y, Roizman B & Weichselbaum R R (1998) Enhancement of Replication of Genetically Engineered Herpes Simplex Viruses by Ionizing Radiation: A New Paradigm for Destruction of Therapeutically Intractable Tumors. Gene Ther 5:160-165.
Akiyama T, Ishida J, Nakagawa S, Ogawara H, Watanabe S, Itoh N, Shibuya M & Fukami Y (1987) Genistein, a Specific Inhibitor of Tyrosine-Specific Protein Kinases. J Biol Chem 262:5592-5595.
Allam A, Taghian A, Gioioso D, Duffy M & Suit H D (1993) Intratumoral Heterogeneity of Malignant Gliomas Measured in Vitro. Int J Radiat Oncol Biol Phys 27:303-308.
Alon T, Hemo I, Itin A, Pe'er J, Stone J & Keshet E (1995) Vascular Endothelial Growth Factor Acts as a Survival Factor for Newly Formed Retinal Vessels and Has Implications for Retinopathy of Prematurity. Nat Med 1:1024-1028.
Altschul S F, Gish W, Miller W, Myers E W & Lipman D J (1990) Basic Local Alignment Search Tool. J Mol Biol 215:403-410.
Antonakopoulos G N, Darnton S J, Newman J, Duffy J P & Matthews H R (1994) Effects of Chemotherapy on Ultrastructure of Oesophageal Squamous Cell Carcinoma. Histopathology 25:447-454.
Arap W, Pasqualini R & Ruoslahti E (1998) Cancer Treatment by Targeted Drug Delivery to Tumor Vasculature in a Mouse Model. Science 279:377-380.
Ausubel F M (ed.) (1995) Short Protocols in Molecular Biology, 3rd ed. Wiley, New York, United States of America.
Baher A G, Andres M L, Folz-Holbeck J, Cao J D & Gridley D S (1999) A Model Using Radiation and Plasmid-Mediated Tumor Necrosis Factor-Alpha Gene Therapy for Treatment of Glioblastomas. Anticancer Res 19:2917-2924.
Baillie C T, Winslet M C & Bradley N J (1995) Tumour Vasculature—a Potential Therapeutic Target. Br J Cancer 72:257-267.
Barton G J (1998) Protein Sequence Alignment Techniques. Acta Crystallogr D Biol Crystallogr 54:1139-1146.
Batzer M A, Cariton J E & Deininger P L (1991) Enhanced Evolutionary Pcr Using Oligonucleotides with Inosine at the 3′-Terminus. Nucleic Acids Res 19:5081.
Baumann M, DuBois W, Pu A, Freeman J & Suit H D (1992) Response of Xenografts of Human Malignant Gliomas and Squamous Cell Carcinomas to Fractionated Irradiation. Int J Radiat Oncol Biol Phys 23:803-809.
Bauminger S & Wilchek M (1980) The Use of Carbodiimides in the Preparation of Immunizing Conjugates. Methods Enzymol 70:151-159.
Becerril B, Poul M A & Marks J D (1999) Toward Selection of Internalizing Antibodies from Phage Libraries. Biochem Biophys Res Commun 255:386-393.
Bell C, Lynam E, Landfair D J, Janjic N & Wiles M E (1999) Oligonucleotide Nx1838 Inhibits Vegf165-Mediated Cellular Responses in Vitro. In Vitro Cell Dev Biol Anim 35:533-542.
Betageri G V, Jenkins S A & Parsons D (eds.) (1993) Liposome Drug Delivery Systems. Technomic Publishing, Lancaster; Pa., United States of America.
Bradford M M (1976) A Rapid and Sensitive Method for the Quantitation of Microgram Quantities of Protein Utilizing the Principle of Protein-Dye Binding. Anal Biochem 72:248-254.
Brognard J, Clark A, Ni Y & Dennis P (2001) Akt/Protein Kinase B Is Constitutively Active in Non-Small Cell Lung Cancer Cells and Promotes Cellular Survival and Resistance to Chemotherapy and Radiation. Cancer Res 61:3986-3997.
Brunelle J K & Chandel N S (2002) Oxygen deprivation induced cell death: An update, Apoptosis 7:475-82.
Burg M A, Pasqualini R, Arap W, Ruoslahti E & Stallcup W B (1999) Ng2 Proteoglycan-Binding Peptides Target Tumor Neovasculature. Cancer Res 59:2869-2874.
Cantley L (2002) The Phosphoinositide 3-Kinase Pathway. Science 296:1655-1657.
Cardone M, Roy N, Stennicke H R, Salvesen G S, Franke T F, Stanbridge E, Frisch S & Reed J C (1998) Regulation of Cell Death Protease Caspase-9 by Phosphorylation. Science 282:1318-1321.
Cheng J, Ruggeri B, Klein W, Sonoda G, Altomare D, Watson D & Testa J (1996) Amplification of Akt2 in Human Pancreatic Cells and Inhibition of Akt2 Expression and Tumorigenicity by Antisense Rna. Proc Natl Acad Sci USA 93:3636-3641.
Datta S, Brunet A & Greenberg M (1999) Cellular Survival: A Play in Three Akts. Genes Dev 13:2905-2927.
Datta S, Dudek H, Tao X, Masters S, Fu H, Gotoh Y & Greenberg M (1997) Akt Phosphorylation of Bad Couples Survival Signals to the Cell-Intrinsic Death Machinery. Cell 91:231-241.
Dracopoli N C, Haines J L, Korf B R, Morton C C, Seidman C E, Seidman J G & Smith D R (eds.) (1997) Current Protocols in Human Genetics on CD-ROM. John Wiley & Sons, New York, United States of America.
Dudek H, Datta S R, Franke T F, Birnbaum M J, Yao R, Cooper G M, Segal R A, Kaplan D R & Greenberg M E (1997) Regulation of Neuronal Survival by the Serine-Threonine Protein Kinase Akt. Science 275:661-665.
Ellerby H M, Arap W, Ellerby L M, Kain R, Andrusiak R, Rio G D, Krajewski S, Lombardo C R, Rao R, Ruoslahti E, Bredesen D E & Pasqualini R (1999) Anti-Cancer Activity of Targeted Pro-Apoptotic Peptides. Nat Med 5:1032-1038.
European Patent No. 0 439 095
Fang X, Yu S, LaPushin R, Lu Y, Furui T, Penn L Z, Stokoe D, Erickson J R, Bast R C, Jr. & Mills G B (2000) Lysophosphatidic Acid Prevents Apoptosis in Fibroblasts Via G(I)-Protein-Mediated Activation of Mitogen-Activated Protein Kinase. Biochem J 352:135-143.
Fantl W, Johnson D & Williams L (1993) Signalling by Receptor Tyrosine Kinases. Annu Rev Biochem 62:453-481.
Fewell J G, MacLaughlin F, Mehta V, Gondo M, Nicol F, Wilson E & Smith L C (2001) Gene Therapy for the Treatment of Hemophilia B Using Pinc-Formulated Plasmid Delivered to Muscle with Electroporation. Mol Ther 3:574-583.
Filippa N, Sable C L, Filloux C, Hemmings B & Van Obberghen E (1999) Mechanism of Protein Kinase B Activation by Cyclic Amp-Dependent Protein Kinase. Mol Cell Biol 19:4989-5000.
Gao N, Ding M, Zheng J Z, Zhang Z, Leonard S S, Liu K J, Shi X & Jiang B-H (2002) Vanadate Induced Expression of Hypoxia-Inducible Factor 1a and Vascular Endothelial Growth Factor through Phosphatidylinositol 3-Kinase/Akt Pathway and Reactive Oxygen Species. J Biol Chem:M200082200.
Garcia-Barros M, Paris F, Cordon-Cardo C, Lyden D, Rafi S, Haimovitz-Friedman A, Fuks & Kolesnick R (2003) Tumor Response to Radiotherapy Regulated by Endothelial Cell Apoptosis. Science 300:1155.
Geng L, Donnelly E, McMahon G, Lin P C, Sierra-Rivera E, Oshinka H & Hallahan D E (2001) Inhibition of Vascular Endothelial Growth Factor Receptor Signaling Leads to Reversal of Tumor Resistance to Radiotherapy. Cancer Res 61:2413-2419.
Glover D M & Hames B D (1995) DNA Cloning: A Practical Approach, 2nd ed. IRL Press at Oxford University Press, Oxford/New York, United States of America.
Goldman C K, Rogers B E, Douglas J T, Sosnowski B A, Ying W, Siegal G P, Baird A, Campain J A & Curiel D T (1997) Targeted Gene Delivery to Kaposi's Sarcoma Cells Via the Fibroblast Growth Factor Receptor. Cancer Res 57:1447-1451.
Gorski D H, Beckett M A, Jaskowiak N T, Calvin D P, Mauceri H J, Salloum R M, Seetharam S, Koons A, Hari D M, Kufe D W & Weichselbaum R R (1999) Blockage of the Vascular Endothelial Growth Factor Stress Response Increases the Antitumor Effects of Ionizing Radiation. Cancer Res 59:3374-3378.
Graeven U, Fiedler W, Karpinski S, Ergun S, Kilic N, Rodeck U, Schmiegel W & Hossfeld D K (1999) Melanoma-Associated Expression of Vascular Endothelial Growth Factor and Its Receptors Flt-1 and KDR. J Cancer Res Clin Oncol 125:621-629.
Granville D J, Carthy C M, Hunt D W & McManus B (1998) Apoptosis: Molecular Aspects of Cell Death and Disease. Lab Invest 78:893-913.
Green D R & Reed J C (1998) Mitochondria and Apoptosis. Science 281:1309-1312.
Gregoriadis G (ed) (1993) Liposome Technology, 2nd ed. CRC Press, Boca Raton, Fla., United States of America.
Grugel S, Finkenzeller G, Weindel K, Barleon B & Marme D (1995) Both V-Ha-Ras and V-Raf Stimulate Expression of the Vascular Endothelial Growth Factor in NIH 3T3 Cells. J Biol Chem 270:25915-25919.
Hallahan D, Kuchibhotla J & Wyble C (1996) Cell Adhesion Molecules Mediate Radiation-Induced Leukocyte Adhesion to the Vascular Endothelium. Cancer Res 56:5150-5155.
Hallahan D E & Virudachalam S (1999) Accumulation of P-Selectin in the Lumen of Irradiated Blood Vessels. Radiat Res 152:6-13.
Hallahan D E, Staba-Hogan M J, Virudachalam S & Kolchinsky A (1998) X-Ray-Induced P-Selectin Localization to the Lumen of Tumor Blood Vessels. Cancer Res 58:5216-5220.
Hallahan D E, Geng L, Cmelak A J, Chakravarthy A B, Martin W, Scarfone C & Gonzalez A (2001) Targeting Drug Delivery to Radiation-induced Neoantigens in Tumor Microvasculature. J Control Release 74:183-191.
Hallahan D E, Mauceri H J, Seung L P, Dunphy E J, Wayne J D, Hanna N N, Toledano A, Hellman S, Kufe D W & Weichselbaum R R (1995) Spatial and Temporal Control of Gene Therapy Using Ionizing Radiation. Nat Med 1:786-791.
Hara K, Yonezawa K, Sakaue H, Ando A, Kotani K, Kitamura T, Kitamura Y, Ueda H, Stephens L, Jackson T, Waterfield M & Kasuga M (1994) 1-Phosphatidylinositol 3-Kinase Activity Is Required for Insulin-Stimulated Glucose Transport but Not for Ras Activation in Cho Cells. Proc Natl Acad Sci USA 91:7415-7419.
Harlow E & Lane D (1988) Antibodies: A Laboratory Manual. Cold Spring Harbor Laboratory Press, Cold Spring Harbor, N.Y., United States of America.
Hawiger J & Timmons S (1992) Binding of Fibrinogen and Von Willebrand Factor to Platelet Glycoprotein IIb-IIIa Complex. Methods Enzymol 215:228-243.
Hawiger J, Kloczewiak M, Bednarek M A & Timmons S (1989) Platelet Receptor Recognition Domains on the Alpha Chain of Human Fibrinogen: Structure-Function Analysis. Biochemistry 28:2909-2914.
Henikoff J G, Pietrokovski S, McCallum C M & Henikoff S (2000) Blocks-Based Methods for Detecting Protein Homology. Electrophoresis 21:1700-1706.
Henikoff S & Henikoff J G (1992) Amino Acid Substitution Matrices from Protein Blocks. Proc Natl Acad Sci USA 89:10915-10919.
Henikoff S & Henikoff J G (2000) Amino Acid Substitution Matrices. Adv Protein Chem 54:73-97.
Hilmas D E & Gillette E L (1975) Microvasculature of C3h/Bi Mouse Mammary Tumors after X-Irradiation. Radiat Res 61:128-143.
Huang C C, Novak W R, Babbitt P C, Jewett A I, Ferrin T E & Klein T E (2000) Integrated Tools for Structural and Sequence Alignment and Analysis. Pac Symp Biocomput:230-241.
Ito T, Qiu H, Collins J A, Brill A B, Johnson D K & Griffin T W (1991) Preclinical Assessments of 90y-Labeled C110 Anti-Carcinoembryonic Antigen Immunotoxin: A Therapeutic Immunoconjugate for Human Colon Cancer. Cancer Res 51:255-260.
Janecki A J, Janecki M, Akhter S & Donowitz M (2000) Basic fibroblast growth factor stimulates surface expression and activity of Na(+)/H(+) exchanger NHE3 via mechanism involving phosphatidylinositol 3-kinase, J Biol Chem 275:8133-8142.
Janoff A S (ed) (1999) Liposomes: Rational Design. M. Dekker, New York, United States of America.
Johnson R (1976) A Thermodynamic Method for Investigation of Radiation Induced Changes in the Microcirculation of Human Tumors. Int J Radiat Oncol Biol Phys 1:659-670.
Kallman R F, DeNardo G L & Stasch M J (1972) Blood Flow in Irradiated Mouse Sarcoma as Determined by the Clearance of Xenon-133. Cancer Res 32:483-490.
Karlin S & Altschul S F (1993) Applications and Statistics for Multiple High-Scoring Segments in Molecular Sequences. Proc Natl Acad Sci USA 90:5873-5877.
Katoh O, Tauchi H, Kawaishi K, Kimura A & Satow Y (1995) Expression of the Vascular Endothelial Growth Factor (VEGF) Receptor Gene, KDR, in Hematopoietic Cells and Inhibitory Effect of VEGF on Apoptotic Cell Death Caused by Ionizing Radiation. Cancer Res 55:5687-5692.
Kauffmann-Zeh A, Rodriguez-Viciana P, Ulrich E, Gilbert C, Coffer P, Downward J & Evan G (1997) Suppression of C-Myc-Induced Apoptosis by Ras Signalling through Pi(3)K and Pkb. Nature 385:544-548.
Kelley T W, Graham M M, Doseff A I, Pomerantz R W, Lau S M, Ostrowski M C, Franke T F & Marsh C B (1999) Macrophage Colony-Stimulating Factor Promotes Cell Survival through Akt/Protein Kinase B. J Biol Chem 274:26393-26398.
Kieser A, Weich H A, Brandner G, Marme D & Kolch W (1994) Mutant P53 Potentiates Protein Kinase C Induction of Vascular Endothelial Growth Factor Expression. Oncogene 9:963-969.
Kirpotin D, Park J W, Hong K, Zalipsky S, Li W L, Carter P, Benz C C & Papahadjopoulos D (1997) Sterically Stabilized Anti-Her2 Immunoliposomes: Design and Targeting to Human Breast Cancer Cells in Vitro. Biochemistry 36:66-75.
Kitamura T, Ogawa W, Sakaue H, Hino Y, Kuroda S, Takata M, Matsumoto M, Maeda T, Konishi H, Kikkawa U & Kasuga M (1998) Requirement for Activation of the Serine-Threonine Kinase Akt (Protein Kinase B) in Insulin Stimulation of Protein Synthesis but Not of Glucose Transport. Mol Cell Biol 18:3708-3717.
Kotani K, Ogawa W, Hino Y, Kitamura T, Ueno H, Sano W, Sutherland C, Granner D K & Kasuga M (1999) Dominant Negative Forms of Akt (Protein Kinase B) and Atypical Protein Kinase Cλ Do Not Prevent Insulin Inhibition of Phosphoenolpyruvate Carboxykinase Gene Transcription. J Biol Chem 274:21305-21312.
Krasilnikov M A (2000) Phosphatidylinositol-3 kinase dependent pathways: the role in control of cell growth, survival, and malignant transformation. Biochemistry (Mosc) 65:59-67.
Kyte J & Doolittle R F (1982) A Simple Method for Displaying the Hydropathic Character of a Protein. J Mol Biol 157:105-132.
Labat-Moleur F, Steffan A M, Brisson C, Perron H, Feugeas O, Furstenberger P, Oberling F, Brambilla E & Behr J P (1996) An Electron Microscopy Study into the Mechanism of Gene Transfer with Lipopolyamines. Gene Ther 3:1010-1017.
Laird A D, Vajkoczy P, Shawver L K, Thurnher A, Liang C, Mohammadi M, Schlessinger J, Ullrich A, Hubbard S R, Blake R A, Fong T A, Strawn L M, Sun L, Tang C, Hawtin R, Tang F, Shenoy N, Hirth K P, McMahon G & Cherrington (2000) Su6668 Is a Potent Antiangiogenic and Antitumor Agent That Induces Regression of Established Tumors. Cancer Res 60:4152-4160.
Lasic D & Martin F (eds.) (1995) STEALTH® Liposomes. CRC Press, Boca Raton, Fla., United States of America.
Lee C G, Heijn M, di Tomaso E, Griffon-Etienne G, Ancukiewicz M, Koike C, Park K R, Ferrara N, Jain R K, Suit H D & Boucher Y (2000) Anti-Vascular Endothelial Growth Factor Treatment Augments Tumor Radiation Response under Normoxic or Hypoxic Conditions. Cancer Res 60:5565-5570.
Leibel S A & Phillips T L (eds.) (1998) Textbook of Radiation Oncology, W B Saunders Company, Philadelphia, Pa., United States of America.
Leunig M, Yuan F, Menger M D, Boucher Y, Goetz A E, Messmer K & Jain R K (1992) Angiogenesis, Microvascular Architecture, Microhemodynamics, and Interstitial Fluid Pressure During Early Growth of Human Adenocarcinoma LS174T in SCID Mice. Cancer Res 52:6553-6560.
Li X, Gong J, Feldman E, Seiter K, Traganos F & Darzynkiewicz Z (1994) Apoptotic Cell Death During Treatment of Leukemias. Leuk Lymphoma 13:65-70.
Lindegaard J C, Overgaard J, Bentzen S M & Pedersen D (1996) Is There a Radiobiologic Basis for Improving the Treatment of Advanced Stage Cervical Cancer? J Natl Cancer Inst Monogr 21:105-112.
Liu B, Earl H, Baban D, Shoaibi M, Fabra A, Kerr D & Seymour L (1995) Melanoma Cell Lines Express VEGF Receptor KDR and Respond to Exogenously Added VEGF. Biochem Biophys Res Commun 217:721-727.
Manome Y, Abe M, Hagen M F, Fine H A & Kufe D W (1994) Enhancer Sequences of the Df3 Gene Regulate Expression of the Herpes Simplex Virus Thymidine Kinase Gene and Confer Sensitivity of Human Breast Cancer Cells to Ganciclovir. Cancer Res 54:5408-5413.
Marin M, Noel D & Piechaczyk M (1997) Towards Efficient Cell Targeting by Recombinant Retroviruses. Mol Med Today 3:396-403.
Maruyama-Tabata H, Harada Y, Matsumura T, Satoh E, Cui F, Iwai M, Kita M, Hibi S, Imanishi J, Sawada T & Mazda 0 (2000) Effective Suicide Gene Therapy in Vivo by Ebv-Based Plasmid Vector Coupled with Polyamidoamine Dendrimer. Gene Ther 7:53-60.
McMahon G (2000) VEGF Receptor Signaling in Tumor Angiogenesis. Oncologist 5:3-10.
Mendel D B, Laird A D, Xin X, Louie S G, Christensen J G, Li G, Schreck R E, Abrams T J, Ngai T J, Lee L B, Murray L J, Carver J, Chan E, Moss K G, Haznedar J O, Sukbunthemg J, Blake R A, Sun L, Tang C, Miller T, Shirazian S, McMahon G & Cherrington J M (2003) In vivo antitumor activity of SU11248, a novel tyrosine kinase inhibitor targeting vascular endothelial growth factor and platelet-derived growth factor receptors: determination of a pharmacokinetic/pharmacodynamic relationship. Clin Cancer Res 9:327-37.
Mesner P W, Jr., Budihardjo, I I & Kaufmann S H (1997) Chemotherapy-Induced Apoptosis. Adv Pharmacol 41:461-499.
Needleman S B & Wunsch C D (1970) A General Method Applicable to the Search for Similarities in the Amino Acid Sequence of Two Proteins. J Mol Biol 48:443-453.
Neri D, Carnemolla B, Nissim A, Leprini A, Querze G, Balza E, Pini A, Tarli L, Halin C, Neri P, Zardi L & Winter G (1997) Targeting by Affinity-Matured Recombinant Antibody Fragments of an Angiogenesis Associated Fibronectin Isoform. Nat Biotechnol 15:1271-1275.
Ohtsuka E, Matsuki S, Ikehara M, Takahashi Y & Matsubara K (1985) An Alternative Approach to Deoxyoligonucleotides as Hybridization Probes by Insertion of Deoxyinosine at Ambiguous Codon Positions. J Biol Chem 260:2605-2608.
Packer R J (1999) Brain Tumors in Children. Arch Neurol 56:421-425.
Paris F, Fuks Z, Kang A, Capodieci P, Juan G, Ehleiter D, Haimovitz-Friedman A, C C-C & R. K (2001) Endothelial Apoptosis as the Primary Lesion Initiating Intestinal Radiation Damage in Mice. Science 293:293-297.
Park J W, Hong K, Kirpotin D B, Meyer O, Papahadjopoulos D & Benz C C (1997) Anti-Her2 Immunoliposomes for Targeted Therapy of Human Tumors. Cancer Lett 118:153-160.
Pasqualini R & Ruoslahti E (1996) Organ Targeting in Vivo Using Phage Display Peptide Libraries. Nature 380:364-366.
Pasqualini R, Koivunen E & Ruoslahti E (1997) Alpha V Integrins as Receptors for Tumor Targeting by Circulating Ligands. Nat Biotechnol 15:542-546.
PCT International Publication No. WO 98/10795.
Pearson W R & Lipman D J (1988) Improved Tools for Biological Sequence Comparison. Proc Natl Acad Sci USA 85:2444-2448.
Powis G, Bonjouklian R, Berggren M, Gallegos A, Abraham R, Ashendel C, Zalkow L, Matter W, Dodge J & Grindey G (1994) Wortmannin, a Potent and Selective Inhibitor of Phosphatidylinositol-3-Kinase. Cancer Res 54:2419-2423.
Radisavljevic Z, Avraham H & Avraham S (2000) Vascular Endothelial Growth Factor up-Regulates Icam-1 Expression Via the Phosphatidylinositol 3 Oh-Kinase/Akt/Nitric Oxide Pathway and Modulates Migration of Brain Microvascular Endothelial Cells. J Biol Chem 275:20770-20774.
Rosenzweig K E, Youmell M B, Palayoor S T & Price B D (1997) Radiosensitization of Human Tumor Cells by the Phosphatidylinositol3-Kinase Inhibitors Wortmannin and Ly294002 Correlates with Inhibition of DNA-Dependent Protein Kinase and Prolonged G2-M Delay. Clin Cancer Res 3:1149-1156.
Rossolini G M, Cresti S, Ingianni A, Cattani P, Riccio M L & Satta G (1994) Use of Deoxyinosine-Containing Primers Vs Degenerate Primers for Polymerase Chain Reaction Based on Ambiguous Sequence Information. Mol Cell Probes 8:91-98.
Sakaue H, Ogawa W, Takata M, Kuroda S, Kotani K, Matsumoto M, Sakaue M, Nishio S, Ueno H & Kasuga M (1997) Phosphoinositide 3-Kinase Is Required for Insulin-Induced but Not for Growth Hormone- or Hyperosmolarity-Induced Glucose Uptake in 3t3-L1 Adipocytes. Mol Endocrinol 11:1552-1562.
Saltzman W M & Fung L K (1997) Polymeric Implants for Cancer Chemotherapy. Adv Drug Deliv Rev 26:209-230.
Sambrook J, Fritsch, E F, and Maniatis, T, eds. (1989) Molecular Cloning: A Laboratory Manual (Second Edition), Cold Spring Harbor Laboratory Press, Cold Spring Harbor, N.Y., United States of America.
Saqi M A, Wild D L & Hartshom M J (1999) Protein Analyst—a Distributed Object Environment for Protein Sequence and Structure Analysis. Bioinformatics 15:521-522.
Shaw M, Cohen P & Alessi D R (1998) The Activation of Protein Kinase B by H2o2 or Heat Shock Is Mediated by Phosphoinositide 3-Kinase and Not by Mitogen-Activated Protein Kinase-Activated Protein Kinase-2. Biochem J 336:241-246.
Shintani S, Kiyota A, Mihara M, Nakahara Y, Terakado N, Ueyama Y & Matsumura T (2000) Association of Preoperative Radiation Effect with Tumor Angiogenesis and Vascular Endothelial Growth Factor in Oral Squamous Cell Carcinoma. Jpn J Cancer Res 91:1051-1057.
Silhavy T J, Berman M L, Enquist L W & Cold Spring Harbor Laboratory (1984) Experiments with Gene Fusions. Cold Spring Harbor Laboratory, Cold Spring Harbor, N.Y., United States of America.
Smith T F & Waterman M (1981) Comparison of Biosequences. Adv Appl Math 2:482-489.
Somervaille T C, Linch D C & Khwaja A (2001) Growth Factor Withdrawal from Primary Human Erythroid Progenitors Induces Apoptosis through a Pathway Involving Glycogen Synthase Kinase-3 and Bax. Blood 98:1374-1381.
Song C W, Payne J T & Levitt S H (1972) Vascularity and Blood Flow in X-Irradiated Walker Carcinoma 256 of Rats. Radiology 104:693-697.
Staba M J, Mauceri H J, Kufe D W, Hallahan D E & Weichselbaum R R (1998) Adenoviral Tnf-Alpha Gene Therapy and Radiation Damage Tumor Vasculature in a Human Malignant Glioma Xenograft. Gene Ther 5:293-300.
Suit H (1996) Assessment of the Impact of Local Control on Clinical Outcome. Front Radiat Ther Oncol 29:17-23.
Taghian A, Ramsay J, Allalunis-Turner J, Budach W, Gioioso D, Pardo F, Okunieff P, Bleehen N, Urtasun R & Suit H (1993) Intrinsic Radiation. Sensitivity May Not Be the Major Determinant of the Poor Clinical Outcome of Glioblastoma Multiforme. Int J Radiat Oncol Biol Phys 25:243-249.
Tam P, Monck M, Lee D, Ludkovski O, Leng E C, Clow K, Stark H, Scherrer P, Graham R W & Cullis P R (2000) Stabilized Plasmid-Lipid Particles for Systemic Gene Therapy. Gene Ther 7:1867-1874.
Taneja N, Tjalkens R, Philbert M A & Rehemtulla A (2001) Irradiation of mitochondria initiates apoptosis in a cell free system, Oncogene 20:167-77
Ting L L, Belfi C A, Tefft M & Ngo F Q (1991) Kht Sarcoma Blood Perfusion Change after Single-Dose X-Ray Irradiation. Int J Radiat Biol 60:335-339.
Tijssen P (1993) Laboratory Techniques in Biochemistry and Molecular Biology-Hybridization with Nucleic Acid Probes, part I chapter 2, Elsevier, New York, N.Y., United States of America.
Ushio-Fukai M, Alexander R W, Akers M, Yin Q, Fujio Y, Walsh K & Griendling K K (1999) Reactive Oxygen Species Mediate the Activation of Akt/Protein Kinase B by Angiotensin li in Vascular Smooth Muscle Cells. J Biol Chem 274:22699-22704.
U.S. Pat. No. 4,196,265
U.S. Pat. No. 4,235,871
U.S. Pat. No. 4,551,482
U.S. Pat. No. 4,554,101
U.S. Pat. No. 4,946,778
U.S. Pat. No. 5,011,634
U.S. Pat. No. 5,091,513
U.S. Pat. No. 5,111,867
U.S. Pat. No. 5,132,405
U.S. Pat. No. 5,260,203
U.S. Pat. No. 5,480,906
U.S. Pat. No. 5,490,840
U.S. Pat. No. 5,510,103
U.S. Pat. No. 5,574,172
U.S. Pat. No. 5,632,991
U.S. Pat. No. 5,651,991
U.S. Pat. No. 5,667,988
U.S. Pat. No. 5,677,427
U.S. Pat. No. 5,688,931
U.S. Pat. No. 5,702,892
U.S. Pat. No. 5,714,166
U.S. Pat. No. 5,780,225
U.S. Pat. No. 5,786,387
U.S. Pat. No. 5,840,479
U.S. Pat. No. 5,849,877
U.S. Pat. No. 5,854,027
U.S. Pat. No. 5,855,900
U.S. Pat. No. 5,858,410
U.S. Pat. No. 5,892,019
U.S. Pat. No. 5,922,254
U.S. Pat. No. 5,922,356
U.S. Pat. No. 5,922,545
U.S. Pat. No. 5,948,647
U.S. Pat. No. 5,948,767
U.S. Pat. No. 5,985,279
U.S. Pat. No. 5,994,392
U.S. Pat. No. 6,054,561
U.S. Pat. No. 6,056,938
U.S. Pat. No. 6,057,098
U.S. Pat. No. 6,071,890
U.S. Pat. No. 6,090,925
U.S. Pat. No. 6,106,866
U.S. Pat. No. 6,120,787
U.S. Pat. No. 6,127,339
U.S. Pat. No. 6,132,766
U.S. Pat. No. 6,174,708
U.S. Pat. No. 6,180,084
U.S. Pat. No. 6,190,700
U.S. Pat. No. 6,197,333
U.S. Pat. No. 6,210,707
U.S. Pat. No. 6,217,886
U.S. Pat. No. 6,221,958
U.S. Pat. No. 6,238,704
U.S. Pat. No. 6,238,705
U.S. Pat. No. 6,245,740
U.S. Pat. No. 6,262,127
U.S. Pat. No. 6,267,981
U.S. Pat. No. 6,287,587
U.S. Pat. No. 6,296,832
U.S. Pat. No. 6,296,842
U.S. Pat. No. 6,312,713
U.S. Pat. No. 6,335,035
Valter M M, Hugel A, Huang H J, Cavenee W K, Wiestler O D, Pietsch T & Wernert N (1999) Expression of the Ets-1 Transcription Factor in Human Astrocytomas Is Associated with Fms-Like Tyrosine Kinase-1 (Fit-1)/Vascular Endothelial Growth Factor Receptor-1 Synthesis and Neoangiogenesis. Cancer Res 59:5608-5614.
Vlahos C J, Matter W F, Hui K Y & Brown R F (1994) A Specific Inhibitor of Phosphatidylinositol 3-Kinase, 2-(4-Morpholinyl)-8-Phenyl-4h-1-Benzopyran-4-One (Ly294002). J Biol Chem 269:5241-5248.
Walker M D, Green S B, Byar D P, Alexander E, Jr., Batzdorf U, Brooks W H, Hunt W E, MacCarty C S, Mahaley M S, Jr., Mealey J, Jr., Owens G, Ransohoff J, 2nd, Robertson J T, Shapiro W R, Smith K R, Jr., Wilson C B & Strike T A (1980) Randomized Comparisons of Radiotherapy and Nitrosoureas for the Treatment of Malignant Glioma after Surgery. N Engl J Med 303:1323-1329.
Wallner K E, Galicich J H, Krol G, Arbit E & Malkin M G (1989) Patterns of Failure Following Treatment for Glioblastoma Multiforme and Anaplastic Astrocytoma. Int J Radiat Oncol Biol Phys 16:1405-1409.
Wang D, Huang H-JS, Kazlauskas A & Cavenee W K (1999) Induction of Vascular Endothelial Growth Factor Expression in Endothelial Cells by Platelet-Derived Growth Factor through the Activation of Phosphatidylinositol 3-Kinase. Cancer Res 59:1464-1472.
Weiner J A & Chun J (1999) Schwann Cell Survival Mediated by the Signaling Phospholipid Lysophosphatidic Acid. Proc Natl Acad Sci USA 96:5233-5238.
Weisberg E, Boulton C, Kelly L M, Manley P, Fabbro D, Meyer T, Gilliland D G & Griffin J D (2002) Inhibition of Mutant Flt3 Receptors in Leukemia Cells by the Small Molecule Tyrosine Kinase Inhibitor Pkc412. Cancer Cell 1:433-443.
Wymann M & Pirola L (1998) Structure and Function of Phosphoinositide 3-Kinases. Biochim Biophys Acta 1436:127-150.
Yamaura H, Yamada K & Matsuzawa T (1976) Radiation Effect on the Proliferating Capillaries in Rat Transparent Chambers. Int J Radiat Biol Relat Stud Phys Chem Med 30:179-187.
Yano S, Tokumitsu H & Soderling T R (1998) Calcium Promotes Cell Survival through Cam-K Kinase Activation of the Protein-Kinase-B Pathway. Nature 396:584-587.
Yao R & Cooper G M (1995) Requirement for Phosphatidylinositol-3 Kinase in the Prevention of Apoptosis by Nerve Growth Factor. Science 267:2003-2006.

It will be understood that various details of the presently claimed subject matter can be changed without departing from the scope of the presently claimed subject matter. Furthermore, the foregoing description is for the purpose of illustration only, and not for the purpose of limitation—the presently claimed subject matter being defined by the claims.

What is claimed is:

1. A method for increasing the radiosensitivity of a target tissue in a subject comprising administering a PI3K antagonist to the subject, whereby the radiosensitivity of the target tissue is increased.

2. The method of claim 1, wherein the target tissue is endothelial tissue.

3. The method of claim 2 wherein the endothelial tissue is vascular endothelium.

4. The method of claim 1, wherein the target tissue is a tumor.

5. The method of claim 4, wherein the tumor comprises a radiation resistant tumor.

6. The method of claim 1, wherein the target tissue comprises vasculature supplying blood flow to a tumor.

7. The method of claim 1, wherein the subject is a mammal.

8. The method of claim 1, wherein the administering a PI3K antagonist comprises administering a minimally therapeutic dose of a PI3K antagonist.

9. The method of claim 1, wherein the administering comprises administering a composition comprising:
(a) a PI3K antagonist; and
(b) a pharmaceutically acceptable carrier.

10. The method of claim 1, wherein the PI3K antagonist comprises Wortmannin.

11. The method of claim 10, wherein the Wortmannin is administered in an amount ranging from about 1 to about 1000 mg/kg.

12. The method of claim 1, wherein the PI3K antagonist comprises LY294002.

13. The method of claim 12, wherein the LY294002 is administered in an amount ranging from 1 to about 1000 mg/kg.

14. The method of claim 1, wherein the PI3K antagonist is a dominant negative PI3K polypeptide.

15. The method of claim 1, wherein the PI3K antagonist is SU6668.

16. The method of claim 1, wherein the PI3K antagonist is SU11248.

17. The method of claim 1, wherein the PI3K antagonist is Genistein.

18. A method for suppressing tumor growth in a subject, the method comprising:
(a) administering a PI3K antagonist to a subject bearing a tumor to increase the radiosensitivity of the tumor; and
(b) treating the tumor with ionizing radiation, whereby tumor growth is suppressed.

19. The method of claim 18, wherein the subject is a mammal.

20. The method of claim 18, wherein the administering a PI3K antagonist comprises administering a minimally therapeutic dose of a PI3K antagonist.

21. The method of claim 18, wherein the administering a PI3K antagonist comprises administering a composition comprising:
(a) a PI3K antagonist; and
(b) a pharmaceutically acceptable carrier.

22. The method of claim 18, wherein the PI3K antagonist comprises Wortmannin.

23. The method of claim 22, wherein the Wortmannin is administered in an amount ranging from 1 to about 1000 mg/kg.

24. The method of claim 18, wherein the PI3K antagonist comprises LY294002.

25. The method of claim 24, wherein the LY294002 is administered in an amount ranging from 1 to about 1000 mg/kg.

26. The method of claim 18, wherein the PI3K antagonist is a dominant negative PI3K polypeptide.

27. The method of claim 18, wherein the PI3K antagonist is SU6668.

28. The method of claim 18, wherein the PI3K antagonist is SU11248.

29. The method of claim 18, wherein the PI3K antagonist is Genistein.

30. The method of claim 18, wherein the tumor comprises a radiation resistant tumor.

31. The method of claim 18, wherein the treating the tumor with ionizing radiation comprises treating the tumor with a subtherapeutic dose of ionizing radiation.

32. A method for inhibiting tumor blood vessel growth, the method comprising:
(a) administering a PI3K antagonist to a subject bearing a tumor to increase the radiosensitivity of tumor blood vessels; and
(b) treating the tumor with ionizing radiation, whereby tumor blood vessel growth is inhibited.

33. The method of claim 32, wherein the administering a PI3K antagonist comprises administering a minimally therapeutic dose of a PI3K antagonist.

34. The method of claim 32, wherein the administering a PI3K antagonist comprises administering a composition comprising:
(a) a PI3K antagonist; and
(b) a pharmaceutically acceptable carrier.

35. The method of claim 32, wherein the PI3K antagonist comprises Wortmannin.

36. The method of claim 35, wherein the Wortmannin is administered in an amount raging from 1 to about 1000 mg/kg.

37. The method of claim 32, wherein the PI3K antagonist comprises LY294002.

38. The method of claim 37, wherein the LY294002 is administered in an amount raging from 1 to about 1000 mg/kg.

39. The method of claim 32, wherein the PI3K antagonist is a dominant negative PI3K polypeptide.

40. The method of claim 32, wherein the PI3K antagonist is SU6668.

41. The method of claim 32, wherein the PI3K antagonist is SU11248.

42. The method of claim 32, wherein the PI3K antagonist is Genistein.

43. The method of claim 32, wherein the subject is a mammal.

44. The method of claim 32, wherein the tumor comprises a radiation resistant tumor.

45. The method of claim 32, wherein the treating the tumor with ionizing radiation comprises treating the tumor with a subtherapeutic dose of ionizing radiation.

46. The method of claim 32, further comprising reducing the vascular length density of the tumor blood vessels.
